SEQUENCE LISTING (4 sequences)

SEQ ID NO: 1 - protein, 10 aa, Homo sapiens
  MOD_RES 10: note = N-methylmethionine
DMHDFFVGLM

SEQ ID NO: 2 - protein, 6 aa, synthetic construct
  MOD_RES 3: note = N-methylphenylalanine
  MOD_RES 6: note = N-methylmethionine
DFFGLM

SEQ ID NO: 3 - DNA, 65 bp, Mus musculus
  source 1..65: mol_type = genomic DNA
gatccgccgcctcaaccccatagcaattagaagcttgtaattgctatggggttgaggcttttttt

SEQ ID NO: 4 - protein, 65 aa, Mus musculus
CTAGAAAAAAAGCCTCAACCCCATAGCAATTACAAGCTTCTAATTGCTATGGGGTTGAGGCGGCG

(19) United States
(12) Patent Application Publication
(10) Pub. No.: US 2026/0048145 A1
(43) Pub. Date: Feb. 19, 2026

(54) COMPOSITIONS AND METHODS FOR TREATING DRUG ADDICTION

(71) Applicant: The Children's Medical Center Corporation, Boston, MA (US)

(72) Inventors: Yi Zhang, Newton, MA (US); Zhengdong Zhao, Brookline, MA (US)

(73) Assignee: The Children's Medical Center Corporation, Boston, MA (US)

(21) Appl. No.: 19/102,477

(22) PCT Filed: Aug. 10, 2023
(86) PCT No.: PCT/US2023/071993

§ 371 Date: Feb. 10, 2025

Related U.S. Application Data
(60) Provisional application No. 63/397,241, filed on Aug. 11, 2022.

(51) Int. Cl. A61K 48/00; A61K 9/00; A61K 31/5513; A61K 38/08; A61K 38/17; A61P 25/30; C12N 15/86

(52) U.S. Cl. CPC A61K 48/005; A61K 9/0019; A61K 31/5513; A61K 38/08; A61K 38/177; A61K 48/0075; A61P 25/30; C12N 15/86; C12N 2750/14143

(57) ABSTRACT

Provided herein are methods for treating drug addiction in a subject, the method comprising activating neurokinin B (NKB)-expressing neurons of the subject. Also provided herein are compositions useful for the treatment of drug addiction in a subject by activating neurokinin B (NKB)-expressing neurons.

### RELATED APPLICATION

This application claims the benefit under 35 U.S.C. § 119 (e) to U.S. Provisional Application No. 63/397,241, entitled “COMPOSITIONS AND METHODS FOR TREATING DRUG ADDICTION”, filed Aug. 11, 2022, the entire contents of which are incorporated herein by reference.

### GOVERNMENT SUPPORT

This invention was made with government support under 5R01DA042283 awarded by the National Institutes of Health. The government has certain rights in the invention.

### REFERENCE TO AN ELECTRONIC SEQUENCE LISTING

The contents of the electronic sequence listing (C123370254WO00-SEQ-VLJ.xml; Size: 5,765 bytes; and Date of Creation: Jul. 31, 2023) is herein incorporated by reference in its entirety.

### BACKGROUND OF INVENTION

Drug addiction is a chronic, relapsing disease with enormous health, social and economic consequences. The striatum plays a critical role in regulating addiction-related behaviors, while the nucleus accumbens (NAc) specifically plays a critical role in processing the reward and motivation related information that regulates addiction related behaviors. The conventional dichotomy model suggests that striatal dopamine 1 receptor (D1) and dopamine 2 receptor (D2) medium spiny neurons (MSNs) positively and negatively regulate addiction-related behaviors, respectively. However, it remains unclear whether there are MSN subtypes beyond the pan-D1/D2 populations that play distinct roles during drug addiction, especially in regard to reward behavior.

### SUMMARY OF INVENTION

The present disclosure is based on the discovery that activation of neurokinin B (NKB)-expressing neurons, specifically NKB-expressing dopamine 1 receptor neurons located in the nucleus accumbens, negatively regulates addiction reward and restores NKB-expressing neuronal activity that is suppressed during drug addiction. Recognition of this phenomenon can be used to better treat drug addiction by directly reducing reward behavior occurring in the striatum of patients. Accordingly, some aspects of the present disclosure relate to a method of treating drug addiction, the method comprising activating NKB-expressing neurons in a subject.

In some embodiments, the NKB-expressing neurons are dopamine 1 receptor (D1) neurons. In some embodiments, the NKB-expressing neurons are medium spiny neurons. In some embodiments, the NKB-expressing neurons are located in the nucleus accumbens (NAc) of the subject.

In some embodiments, the method comprises administering to the subject an effective amount of an agent for stimulating activity of the NKB-expressing neurons in the subject.

In some embodiments, the agent is a small molecule, a hormone, a protein, a peptide, and aptamer, or a nucleic acid.

In some embodiments, the agent activates a G-protein coupled receptor expressed in NKB-expressing neurons of the subject. In some embodiments, the agent is an agonist of the G-protein coupled receptor. In some embodiments, the G-protein coupled receptor is selected from Neurokinin 3 Receptor (NK3R), Thyrotropin Releasing Hormone Receptor (TRHR), and G-Protein Coupled Receptor 158 (GPR158). In some embodiments, the G-protein coupled receptor is NK3R and the agent is senktide.

In some embodiments, the agent is a nucleic acid encoding an active form of a G-protein coupled receptor. In some embodiments, the G-protein coupled receptor is selected from NK3R, TRHR, and GPR158.

In some embodiments, the method comprises optogenetically stimulating the activity of the NKB-expressing neurons in the subject. In some embodiments, the method comprises administering to the subject a vector encoding an optogenetically activated protein and laser-stimulating the activity of the optogenetically activated protein in NKB-expressing neurons of the subject. In some embodiments, the optogenetically activated protein is light-gated cation channel channelrhodopsin (ChR2) and the laser stimulation is blue light laser stimulation. In some embodiments, the vector is administered to the NKB-expressing neurons. In some embodiments, the vector is a viral vector. In some embodiments, the viral vector is a recombinant adeno-associated virus (rAAV) vector.

In some embodiments, the method comprises chemogenetically stimulating the activity of the NKB-expressing neurons in the subject. In some embodiments, the method comprises administering to the subject a vector encoding a chemogenetically activated protein and an agent sufficient to activate the chemogenetically activated protein in NKB-expressing neurons of the subject. In some embodiments, the chemogenetically activated protein is a modified human M3 muscarinic receptor (hM3Dq) and the agent is clozapine-N-oxide (CNO). In some embodiments, the vector is administered to the NKB-expressing neurons. In some embodiments, the vector is a viral vector. In some embodiments, the viral vector is a recombinant adeno-associated virus (rAAV) vector.

In some embodiments, the method comprises electrically stimulating activity of the NKB-expressing neurons in the subject. In some embodiments, the method comprises treating NKB-expressing neurons in the subject with deep brain stimulation (DBS).

In some embodiments, the administration occurs via injection. In some embodiments, the administration occurs via intravenous injection, intraperitoneal injection, or intracranial injection.

In some embodiments, the method results in increased signaling from NKB-expressing neurons of the NAc to the lateral hypothalamus. In some embodiments, the method results in decreased drug reward behavior in the subject.

In some embodiments, the subject is a human subject.

In some embodiments, the drug addiction is a drug addiction in which NKB-expressing neuron activity in the subject is reduced. In some embodiments, the drug addiction is selected from nicotine addiction, cocaine addiction, opioid addiction, alcohol addiction, barbiturate addiction, and methamphetamine addiction, or a combination thereof.

Further aspects of the present disclosure provide compositions for use in treating drug addiction in a subject in need thereof, the composition comprising an agent for activating neurokinin B (NKB)-expressing neurons in the subject and a pharmaceutically acceptable excipient.

In some embodiments, the NKB-expressing neurons are dopamine 1 receptor (D1) neurons. In some embodiments, the NKB-expressing neurons are medium spiny neurons. In some embodiments, the NKB-expressing neurons are located in the nucleus accumbens (NAc) of the subject.

In some embodiments, the agent is a small molecule, a hormone, a protein, a peptide, an aptamer, or a nucleic acid.

In some embodiments, the agent activates a G-protein coupled receptor expressed in NKB-expressing neurons of the subject. In some embodiments, the agent is an agonist of the G-protein coupled receptor. In some embodiments, the G-protein coupled receptor is selected from Neurokinin 3 Receptor (NK3R), Thyrotropin Releasing Hormone Receptor (TRHR), and G-Protein Coupled Receptor 158 (GPR158). In some embodiments, the G-protein coupled receptor is NK3R and the agent is senktide.

In some embodiments, the agent is a nucleic acid encoding an active form of a G-protein coupled receptor. In some embodiments, the G-protein coupled receptor is selected from NK3R, TRHR, and GPR158.

In some embodiments, the agent comprises a vector encoding an optogenetically activated protein, wherein laser stimulation activates the optogenetically activated protein, thereby activating NKB-expressing neurons of the subject. In some embodiments, the optogenetically activated protein is light-gated cation channel channelrhodopsin (ChR2) and the laser stimulation is blue light laser stimulation. In some embodiments, the vector is a viral vector. In some embodiments, the viral vector is a recombinant adeno-associated virus (rAAV) vector.

In some embodiments, the agent comprises a vector encoding a chemogenetically activated protein, wherein administration of an agent is sufficient to activate the chemogenetically activated protein, thereby activating NKB-expressing neurons of the subject. In some embodiments, the chemogenetically activated protein is a modified human M3 muscarinic receptor (hM3Dq) and the agent is clozapine-N-oxide (CNO). In some embodiments, the vector is a viral vector. In some embodiments, the viral vector is a recombinant adeno-associated virus (rAAV) vector.

In some embodiments, the composition is suitable for administration via injection. In some embodiments, the injection comprises intravenous injection or intracranial injection.

In some embodiments, administration of the composition to the subject results in increased signaling from NKB-expressing neurons of the NAc to the lateral hypothalamus. In some embodiments, administration of the composition to the subject results in decreased drug reward behavior in the subject.

In some embodiments, the subject is a human subject.

In some embodiments, the drug addiction is a drug addiction in which NKB-expressing neuron activity in the subject is reduced. In some embodiments, the drug addiction is selected from nicotine addiction, cocaine addiction, opioid addiction, alcohol addiction, barbiturate addiction, and methamphetamine addiction, or a combination thereof.

### BRIEF DESCRIPTION OF DRAWINGS

The accompanying drawings are not intended to be drawn to scale. In the drawings, each identical or nearly identical component that is illustrated in various FIGs. is represented by a like numeral. For purposes of clarity, not every component may be labeled in every drawing.

In the drawings:

FIGS. 1A-1H show single-cell calcium imaging of Tac2+ neurons following saline/cocaine injection. FIG. 1A shows tSNE plot showing the expression of Tac2 in a subpopulation of D1-MSN in NAc. The expression level is color-coded. FIG. 1B shows RNA in situ hybridization showing Tac2 expression in the medial part of the NAc shell. FIG. 1C shows a diagram indicating the locations of the implanted GRIN lenses in the NAc. FIG. 1D shows viral expression of GCaMP6m and lens location in NAc. FIG. 1E shows a pipeline for calcium signal extraction and cell identification. FIG. 1F shows a heatmap of calcium signal traces of all neurons from 6 mice recorded following saline (left) or cocaine (right) injection. The neurons that are excited, inhibited and no response by treatment are shown in the stacked bar graph next to the heatmap. FIG. 1G shows analysis based on calcium trace. Bar graphs showing the percentages of neurons that were excited or inhibited by saline (left) or cocaine (right) administration. (**, p=0.0024; ns, p>0.05, paired t-test). FIG. 1H shows Venn diagrams showing the overlap between the initial phase (0-10 minutes) and late phase (50-60 minutes) of post-injection of cocaine excited neurons (left) or cocaine inhibited neurons (right). Data in FIG. 1G are presented as mean±SEM, scale bars of FIG. 1B and FIG. 1D are indicated in the figures.

FIGS. 2A-21 show Tac2-expressing D1 MSNs in NAc respond to cocaine administration. FIG. 2A shows RNA FISH of Drd1, Drd2, and Tac2. High-magnification images of the boxed areas are on the top right. The Drd1⁺ Tac2⁺ double positive cells account for 97.3±1.5% of Tac2⁺ neurons (n=3 mice, total 548 cells, scale bars 50 μm). FIG. 2B shows a diagram of calcium imaging following saline/cocaine administration. The recorded sessions including the pre-injection phase (−10-0 minutes), the post-injection initial phase (0-10 minutes) and decay phase (50-60 minutes) are indicated. FIG. 2C shows representative calcium traces and transients of individual neurons that were activated or inhibited by cocaine injection. FIG. 2D shows a heatmap of calcium transient frequency of all neurons from 6 mice recorded following saline (left) or cocaine (right) injection. The percentage of neurons that were excited, inhibited or with no response by treatment were shown in the stacked bar graph next to the heatmap. FIG. 2E shows bar graphs showing the percentages of neurons that are excited or inhibited by saline (left) or cocaine (right) administration. FIG. 2F shows a Venn diagram showing the overlap between the initial phase (0-10 minutes) and decay phase (50-60 minutes) of post-injection of cocaine excited neurons (left) or cocaine inhibited neurons (right). FIG. 2G shows cumulative distribution of calcium transient frequencies around saline (left) and cocaine (right) injection. (saline: n=361, cocaine: n=356). FIG. 2H shows a bar graph showing the calcium activity change before and after injection. FIG. 2I shows averaged calcium transient frequency before and after cocaine injection from neurons that showed elevated (left) or reduced (right) activity during the initial phase (0˜10 minutes) of cocaine injection. Data in FIG. 2A, FIG. 2E, FIG. 2H, and FIG. 2I are presented as mean±SEM. *, p≤0.05, **p≤0.01, ****p≤0.0001, ns, p>0.05.

FIGS. 3A-3G show cocaine place conditioning modulates Tac2 neuronal activities that are associated with cocaine-reward contexts. FIG. 3A shows a diagrammatic illustration of the calcium imaging recording experiment during pre- and post-cocaine place conditioning. “S”, conditioning with saline injection; “C”, conditioning with cocaine injection. FIG. 3B shows a bar graph showing the time spent in cocaine chamber during pre- and post-cocaine place conditioning. FIG. 3C upper: representative calcium transients of neurons from one mouse during staying in saline(S) or cocaine (C) coupled chambers. Both the cocaine chamber excited neurons and inhibited neurons are shown. Bottom: averaged calcium traces of the above cocaine chamber excited neurons and inhibited neurons. FIG. 3D upper: pie chart showing the fraction of cells that were excited, inhibited, or no response when staying in a cocaine-associated chamber during pre-conditioning (left) or post-conditioning (right). Bottom: bar graphs showing the percentage of neurons that were excited or inhibited when staying in a cocaine-associated chamber. FIG. 3E shows calcium transient frequency of cocaine chamber excited neurons (left, n=16) and inhibited neurons (right, n=62) during mice staying in saline or cocaine chambers. FIG. 3F left: average calcium transient frequency of cocaine chamber excited neurons before and after entering cocaine chamber. Right: quantification of calcium transient frequency in 5 seconds windows before- and after-entry. FIG. 3G left: average calcium transient frequency of cocaine chamber inhibited neurons before and after entering cocaine chamber. Right: quantification of calcium transient frequency in 5 seconds windows before- and after-entry. Data in FIG. 3B, FIG. 3D, FIG. 3G, and FIG. 3H are presented as mean±SEM, shaded areas in FIG. 3C represent SEM. **p≤0.01, ****P≤0.0001, ns, p>0.05.

FIG. 4 shows percentages of cocaine conditioning responsive neurons in pre- and post-conditioning. Upper: pie charts showing the fractions of Tac2+ neurons that were excited, inhibited, or no response when staying in a cocaine-associated chamber during pre-conditioning (left) or post-conditioning (right). Bottom: bar graphs showing the percentages of neurons that were excited or inhibited when staying in a cocaine-associated chamber during pre-conditioning (left) or post-conditioning (right). *, p=0.044, ns, p>0.05, paired t-test. Data are presented as mean±SEM.

FIGS. 5A-5H shows NAc Tac2⁺ neurons bidirectionally regulate cocaine reward in the cocaine-CPP test. FIG. 5A shows viral expression of ChR2-EYFP and optic cannula placement in NAc. FIG. 5B shows an illustration of the 2-chambers cocaine conditioned place preference (Cocaine-CPP) paradigm. FIG. 5C shows Cocaine-CPP with optogenetic excitation of Tac2⁺ neurons. Left: time spent in the cocaine-paired chambers pre- and post-conditioning. Right: the CPP scores were calculated by subtracting the time spent in pre-conditioning phase from the time spent in post-conditioning phase. FIG. 5D shows the cumulative distance traveled in the 30-minutes of post-treatment period. Mice received saline or cocaine injections and were concurrently given laser stimulation [light pattern: 5×(3 minutes On, 3 minutes Off)]. FIG. 5E shows viral expression of hM3Dq-mCherry in the NAc. FIG. 5F shows Cocaine-CPP with chemogenetic excitation of Tac2+ neurons. Left: time spent in the cocaine-paired chambers pre- and post-conditioning. Right: the CPP scores were calculated by subtracting the time spent in pre-conditioning phase from the time spent in post-conditioning phase. FIG. 5G shows viral expression of hM4Di-mCherry in the NAc. FIG. 5H shows Cocaine-CPP with chemogenetic inhibition of Tac2+ neurons. Left: time spent in the cocaine-paired chambers pre- and post-conditioning. Right: the CPP scores were calculated by subtracting the time spent in pre-conditioning phase from the time spent in post-conditioning phase. Data in FIG. 5C, FIG. 5D, FIG. 5F, and FIG. 5H are presented as mean±SEM, scale bars of FIG. 5A, FIG. 5E, and FIG. 5G are indicated in the figures. *, p≤0.05, **, p≤0.01, ***, p≤0.001, ****, p≤0.0001, ns, p>0.05.

FIGS. 6A-6G show optogenetic activation of NAc Tac2⁺ neurons. FIG. 6A shows laser stimulation (3 cycles of 3 minutes-on, 3 minutes-off) induces strong cFos expression in ChR2-EYFP expressing cells, but not in the control GFP-expressing cells. The ratio of cFos+/GFP+ cells in all GFP+ cells was calculated and shown on the right. ***, p=0.0003, unpaired t-test. FIG. 6B shows diagrams indicating the locations of implanted optic cannulas in the NAc of DIO-ChR2-EYFP expressing mice and DIO-EYFP expressing mice. FIGS. 6C-6G show optogenetic activation of the NAc Tac2-expressing neurons did not affect real-time place preference (FIG. 6C), locomotion in open field arena (FIG. 6D), time spent in the center area of open field arena (FIG. 6E), food intake (FIG. 6F), and elevated plus maze (FIG. 6G). Laser stimulation patterns are indicated in the figures. All p-values were calculated by unpaired t-test, ns, p>0.05.

FIGS. 7A-7D show chemogenetic activation or inhibition of NAc Tac2⁺ neurons. FIG. 7A shows cFos induction after intraperitoneal injection of ligand CNO in hM3Dq-mCherry-expressing and mCherry-expressing mice. The ratio of cFos⁺/mCherry⁺ cells in all mCherry⁺ cells was calculated and shown on the right. ****, p<0.0001, unpaired t-test. FIG. 7B shows cFos induction after intraperitoneal injection of ligand CNO in hM4Di-mCherry-expressing and mCherry-expressing mice subjected to cocaine treatment. The ratio of cFos⁺/mCherry⁺ cells in all mCherry⁺ cells was calculated and shown on the right. **, p=0.002, unpaired t-test. FIG. 7C shows distance traveled in the 1-hour post-treatment period after chemogenetic excitation of Tac2⁺ neurons. **, p≤0.01, ns, p>0.05, unpaired t-test. FIG. 7D shows distance traveled in the 1-hour post-treatment period after chemogenetic inhibition of Tac2⁺ neurons. **, p≤0.01, ns, p>0.05, unpaired t-test. Data are presented as mean±SEM, scale bars of FIG. 7A and FIG. 7B are indicated in the figures.

FIGS. 8A-8I show NAc Tac2⁺ neurons bidirectionally regulate cocaine addiction in the cocaine-IVSA test. FIG. 8A shows diagrammatic illustration of the cocaine intravenous self-administration paradigm (Cocaine-IVSA). Mice were trained to press the lever to get cocaine infusion, pressing the active lever is followed by intravenous cocaine infusion while pressing the inactive lever yields no outcome. The behavioral training includes acquisition phase and dose-responsive curve in the maintenance phase. Mice received CNO injection (2 mg/kg for hM3Dq group and 5 mg/kg for hM4Di group) 20-minutes prior to be placed into self-administration chamber with access to cocaine at doses of 0.03, 0.1, 0.3, or 1 mg/kg/infusion. FIGS. 8B-8E show Cocaine-IVSA with chemogenetic excitation of Tac2⁺ neurons. FIG. 8B shows numbers of lever presses. FIG. 8C shows lever accuracy under the dose of 0.33 mg/kg/infusion. FIG. 8D shows numbers of infusions. FIG. 8E shows cumulative cocaine infusion time courses under the dose of 0.33 mg/kg/infusion. FIGS. 8F-8I show Cocaine-IVSA with chemogenetic inhibition of Tac2⁺ neurons. FIG. 8F shows numbers of lever presses. FIG. 8G shows lever accuracy under the dose of 0.33 mg/kg/infusion. FIG. 8H shows numbers of infusions. FIG. 8I shows cumulative cocaine infusion time courses under the dose of 0.33 mg/kg/infusion. Data in FIGS. 8B-8I are presented as mean±SEM. *, p≤0.05, **, p≤0.01, ***, p<0.001, ns, p>0.05.

FIGS. 9A-9F show intravenous cocaine self-administration. FIGS. 9A-9C show the numbers of active lever press and inactive lever press (FIG. 9A), lever accuracy (FIG. 9B), and numbers of cocaine infusions (FIG. 9C) during the acquisition phase of cocaine IVSA training in response to chemogenetic activation of Tac2⁺ neurons. FIGS. 9D-9F show the numbers of active lever press and inactive lever press (FIG. 9D), lever accuracy (FIG. 9E), and numbers of cocaine infusions (FIG. 9F) during the acquisition phase of cocaine IVSA training in response to chemogenetic inhibition of Tac2⁺ neurons. Data are presented as mean±SEM.

FIGS. 10A-10I show shRNA-mediated knock-down of Tac2 in NAc does not affect cocaine condition place preference nor contingent cocaine taking. FIG. 10A shows representative Tac2 RNA FISH images of mice injected with AAVs expressing Tac2 shRNA or control shRNA. FIG. 10B shows quantification of Tac2 knock-down efficiency. Numbers were calculated by summating Tac2⁺ cells in serial 3 slides of the NAc region of individual mice (˜Bregma+1.2). FIG. 10C shows effects of Tac2 knockdown on cocaine-CPP. Mice were subjected to 2-days conditioning, with each morning given saline injection followed by confined to saline-coupled chamber for 30 minutes, and in the afternoon, mice were given cocaine injection followed by confined to cocaine chamber for 30 minutes. Left: time spent in the cocaine-paired chambers pre- and post-conditioning, ****, p<0.0001, paired t-test. Right: the CPP scores were calculated by subtracting the time spent in pre-conditioning phase from the time spent in post-conditioning phase, ns, p=0.0975, unpaired t-test. FIGS. 10D-10F shows Cocaine-IVSA of Tac2 knockdown mice at acquisition phase. The numbers of active lever press and inactive lever press (FIG. 10D), lever accuracy (FIG. 10E), and numbers of cocaine infusions (FIG. 10F) during the acquisition phase of cocaine IVSA training. FIGS. 10G-10I show dose-dependent response under cocaine-IVSA of Tac2 knockdown mice. The numbers of lever presses (FIG. 10G). Lever accuracy (FIG. 10H), and numbers of infusions (FIG. 10I) are shown. Data in FIGS. 10B-10I are presented as mean±SEM. Scale bars of FIG. 10A are indicated in the figures.

FIGS. 11A-11I show the Tac2⁺ NAc to LH pathway modulates cocaine reward behavior. FIG. 11A shows a diagram of antegrade tracing of the NAc Tac2+ neurons with ChR2-EYFP. FIG. 11B shows AAV-DIO-ChR2-EYFP expression in the NAc injection site. FIGS. 11C-11E show detection of the ChR2-EYFP signals in the nerve terminals in VP (FIG. 11C), LH (FIG. 11D), and VTA (FIG. 11E). FIG. 11F shows the percentages of axon terminal fluorescence signal in VP, LH, and VTA. FIGS. 11G-11I show optogenetic activation of Tac2⁺ NAc to LH projection (FIG. 11H), but not the NAc to VP (FIG. 11G), and NAc to VTA projections (FIG. 11I), reduced the cocaine-CPP score. Data in FIGS. 11F-11I are presented as mean±SEM, scale bars of FIGS. 11B-11E are indicated in the figures. VP: ventral pallidum. LH: lateral hypothalamus. VTA: ventral tegmental area, ac: anterior commissure, BNST: bed nuclei of the stria terminalis, MPO: medial preoptic area, LPO: lateral preoptic area, ZI: zona incerta, DMH: dorsal medial hypothalamus, VMH: ventral medial hypothalamus, IPN: Interpeduncular nucleus, MM: Medial mammillary nucleus, SNr: substantia nigra, reticular part, SNc: substantia nigra, compact part. *, p≤0.05, ns, p>0.05.

FIGS. 12A-12B show systemic injection of a TacR3 agonist, senktide, reduced cocaine-conditioned place preference and cocaine-induced locomotion. FIG. 12A shows Cocaine-CPP with injection of a NKR3 agonist, senktide. The CPP scores were calculated by subtracting the time spent in pre-conditioning phase from the time spent in post-conditioning phase, *, p=0.054, unpaired t-test. FIG. 12B shows distance traveled in the 30-minute post-treatment period after i.p. injection of senktide or injection of saline in cocaine-induced locomotion assay. ***, p=0.0005, unpaired t-test.

### DETAILED DESCRIPTION OF CERTAIN EMBODIMENTS

Aspects of the present disclosure are based on the discovery that neurokinin B (NKB)-expressing dopamine 1 receptor (D1) medium spiny neurons (MSNs), specifically those located in the nucleus accumbens (NAc), play a pivotal role in processing reward and motivation-related behaviors within the striatum. For this reason, changes in NKB-expressing D1-MSN activity are greatly associated with the establishment and continuance of drug addiction. Without wishing to be bound by theory, as addiction is established, activity of NKB-expressing D1-MSNs (which are alternately referred to as Tac2-expressing D1-MSNs in the context of a murine model) is decreased. Unexpectedly, however, stimulation of NKB-expressing D1-MSNs during addiction suppresses addiction-related behaviors. These findings strongly suggest that by directly modulating the activity of D1-MSNs in a subject, it is possible to treat drug addiction at its neurological source. In principle, this approach could be used to treat any addiction in which the normal activity of D1-MSNs is decreased and could be especially useful for the treatment of chronic and relapsing drug addiction for which other treatments have not been successful.

### Methods for Treating Drug Addiction

As used herein, the term “addiction” refers to a disease state that is characterized by excessive activation of reward circuitry occurring in the brain of a subject in response to an external stimulus. The terms “drug addiction” and “substance use disorder” specifically refer to excessive activation of reward circuitry that occurs in response to stimulation by a particular material (a drug or substance). The symptoms used to identify drug addiction in a subject may vary, as addiction to different drugs may tend to produce different symptoms, and different subjects with the same type of drug addiction may display different symptoms. Broadly, symptoms of drug addiction are classified in four groups: symptoms of impaired control (e.g., overuse of a drug, or using more of a drug than intended; use of a drug despite having a desire not to), symptoms of social problems resulting from drug use (e.g., neglecting responsibilities and/or relationships with other individuals, abandoning previous activities of interest due to drug use, inability to complete tasks as a result of drug use), symptoms of risky use (e.g., drug use in high risk settings, continued drug use despite knowing its health hazards), and symptoms of physical dependence (e.g., tolerance to the drug (i.e., requiring more of the drug to achieve the same effect as previously obtained by use of a smaller dose), symptoms of withdrawal when not using the drug). Drug addiction may occur at different levels of severity and typically becomes more severe over time in the absence of treatment. Subjects with a higher severity of drug addiction also tend to demonstrate more symptoms of drug addiction. For example, one symptom may indicate a subject at risk of developing drug addiction, two or three symptoms may indicate a subject with mild drug addiction, four or five symptoms may indicate a subject with moderate drug addiction, and six or more symptoms may indicate a subject with severe drug addiction. Subjects with higher severity drug addiction generally require more intensive treatment in order to reduce excessive activation of reward circuitry. Subjects with higher severity drug addiction may also be at a greater risk of relapse (recurrence of drug addiction) after treatment is withdrawn. Additional information about drug addiction may be found in the “Diagnostic and Statistical Manual of Mental Disorders, Fifth Edition, Text Revision” (2022). United States: American Psychiatric Association Publishing, which is incorporated herein by reference.

As used herein, the terms “drug” and “substance,” in respect to an addiction, may be used interchangeably to refer to a material that possesses addictive properties (i.e., can elicit overactivation of reward circuitry in the brain) when used (e.g., consumed, whether orally, intravenously, or inhaled) by a subject (e.g., a human). Examples of drugs with addictive properties include, but are not limited to, alcohol, caffeine, cannabis, hallucinogens, inhalants, opioids, sedatives, hypnotics, anxiolytics, stimulants, and tobacco.

As used herein, the term “NKB-expressing neuron” refers to a neuronal cell that expresses neurokinin B (NKB), a tachykinin family neuropeptide that is produced from tachykinin 3 precursor protein (NCBI Accession No.: NP_037383.1) and has a sequence set forth as DMHDFFVGLM (SEQ ID NO: 1), which is modified with a carboxy terminal amide group. NKB is encoded by the TAC3 gene in humans and the Tac2 gene in rodents. Without wishing to be bound by theory, NKB interacts with tachykinin 3 receptor (NK3R, encoded by the TAC3R gene in humans), a G-protein coupled receptor on the neuronal cell surface, and are together functionally related to various pathways in both humans and rodents, including the secretion of gonadotropin-releasing hormone (GnRH) and regulation of reproductive cycles (see, e.g., Anderson, et al., “The roles of kisspeptin and neurokinin B in GnRH pulse generation in humans, and their potential clinical application.” J Neuroendocrinol. 2022; 34 (5): e13081, which is incorporated herein by reference). NKB and NK3R have also been implicated in numerous other pathways and disorders, relating to nocireception, mood disorders, schizophrenia, and neurodegeneration (see, e.g., Zhang, et al. “Tacr3/NK3R: Beyond Their Roles in Reproduction.” ACS Chem Neurosci. 2020; 11(19):2935-2943, which is incorporated herein by reference). Within particular regions of the brain, such as the nucleus accumbens (NAc), NKB-expressing neurons, including a subpopulation of neurons that express dopamine 1 receptor (D1), are further involved in the regulation of reward circuitry and may be inactivated as a result of drug use and/or addiction, as exemplified herein.

The activation of NKB-expression neurons may also be related to the function of G-coupled receptors besides NK3R which are expressed on the neuronal cell surface, such as, but not limited to, thyrotropin releasing hormone receptor (TRHR) and G-protein coupled receptor 158 (GPR158). TRHR (NCBI Accession No.: NP_003292.1; Gene ID: 7201) binds to thyrotropin-releasing hormone (TRH), a hypophysiotropic hormone secreted by neurons of the hypothalamus that stimulates the release of thyroid-stimulating hormone (TSH) and prolactin from the anterior pituitary gland. TRH and TRHR are thought to be functionally related to the regulation of mood and metabolism (see, e.g., Alvarez-Salas, et al., “Role of the thyrotropin-releasing hormone of the limbic system in mood and eating regulation.” J Integr Neurosci. 2022 Mar. 18; 21(2):47, which is incorporated herein by reference). GPR158 (NCBI Accession No.: NP_065803.2; Gene ID: 57512) binds to regulator of G-protein signaling 7 (RGS7), a universal inhibitor of Gᵢ alpha subunit-containing G-protein coupled receptors, but is otherwise widely regarded as an orphan G-protein coupled receptor for which an endogenous ligand is not yet known. Like NK3R and TRHR, GPR158 has been shown to be involved in mood regulation and affective disorders (see, e.g., Watkins and Orlandi, “Orphan G Protein Coupled Receptors in Affective Disorders.” Genes (Basel). 2020; 11 (6): 694, which is incorporated herein by reference). The activity of G-protein coupled receptors (e.g., NK3R, TRHR, GPR158) may be modulated by chemical agents, such as, but not limited to a small molecule or a peptide. Such agents may either increase activity of a G-protein coupled receptor (agonist) or decrease activity of a G-protein coupled receptor (antagonist). Without wishing to be bound by theory, NKB-expressing neurons may be activated by treating said neurons with an agent that activates a G-protein coupled receptor located on the NKB-expressing neuron cell surface. As a non-limiting example, NK3R may be activated by treatment with senktide, a tachykinin analog having the sequence Suc-Asp-Phe-(Me-Phe)-Gly-Leu-Met (SEQ ID NO: 2) modified with a carboxy terminal amide group (see, e.g., Misu et al. “Structure-activity relationship study on senktide for development of novel potent neurokinin-3 receptor selective agonists” Med. Chem. Commun., 2015, 6, 469-476, which is incorporated herein by reference).

Accordingly, the present disclosure provides a method for treating drug addiction in a subject, the method comprising activating neurokinin B (NKB)-expressing neurons in the subject. In some embodiments, the NKB-expressing neurons are dopamine 1 receptor (D1)-expressing neurons. In some embodiments, the NKB-expressing neurons are medium spiny neurons. In some embodiments, the NKB-expressing neurons are located in the nucleus accumbens (NAc) of the subject.

In some embodiments, the method comprises administering to the subject an effective amount of an agent for stimulating activity of the NKB-expressing neurons in the subject. The agent may be a small molecule, a hormone, a protein, a peptide, an aptamer, or a nucleic acid. In some embodiments, the agent activates a G-protein coupled receptor expressed in the NKB-expressing neurons of the subject. In some embodiments, the agent is an agonist of the G-protein coupled receptor. In some embodiments, the G-protein coupled receptor is selected from Neurokinin 3 Receptor (NK3R) (NCBI Accession No: NP_001050.1; Gene ID: 6870), Thyrotropin Releasing Hormone Receptor (TRHR) (NCBI Accession No.: NP_003292.1; Gene ID: 7201), and G-Protein Coupled Receptor 158 (GPR158) (NCBI Accession No.: NP_065803.2; Gene ID: 57512). In some embodiments, the agent is an agonist of NK3R, such as, but not limited to, senktide, or a derivative thereof.

In some embodiments, the method comprises ontogenetically stimulating the activity of the NKB-expressing neurons in the subject. Without wishing to be bound by theory, “optogenetic stimulation” of a neuron, as referred to herein, comprises the expression of an “optogenetically activated protein” in the neuron, wherein exposure of the optogenetically activated protein to a particular wavelength of light causes the protein to change conformation and become active, thereby activating the neuron. In some embodiments, the method comprises administering to the subject a vector encoding an optogenetically activated protein and laser-stimulating the activity of the optogenetically activated protein in NKB-expressing neurons of the subject. In some embodiments, the optogenetically activated protein is light-gated cation channel channelrhodopsin (ChR2) and the laser stimulation is blue light laser stimulation. Channelrhodopsins, such as ChR2, are light-mediated ion channels that may be expressed on the surface of neuronal cells and then externally controlled by exposure to particular wavelengths of light (e.g., blue light). Activation of channelrhodopsin proteins on the surface of a neuron causes depolarization and thus activation of the neuron. Techniques for expressing and controlling the activity of optogenetically activated proteins, such as channelrhodopsins, in neuronal cells are well known in the relevant art (see, e.g., Alekseev, et al. “Rhodopsin-Based Optogenetics: Basics and Applications.” Methods Mol Biol. 2022; 2501:71-100, which is incorporated herein by reference). In some embodiments, the optogenetically activated protein is a derivative of ChR2. In some embodiments, the optogenetically activated protein and/or laser stimulation is an alternate optogenetically activated protein and/or laser stimulation that is generally known in the art. In some embodiments, the vector is administered to the NKB-expressing neurons. In some embodiments, the vector is a viral vector. In some embodiments, the viral vector is a recombinant adeno-associated virus (rAAV) vector (e.g., an rAAV vector that is capable of entering neurons located in the NAc), or another suitable viral vector known in the art.

In some embodiments, the method comprises chemogenetically stimulating the activity of the NKB-expressing neurons in the subject. Without wishing to be bound by theory, “chemogenetic stimulation” of a neuron, as referred to herein, comprises the expression of a “chemogenetically activated protein” in the neuron, wherein exposure of the protein to a particular chemical agent causes the protein to change conformation and become active, thereby activating the neuron. In some embodiments, the method comprises administering to the subject a vector encoding a chemogenetically activated protein and a chemical agent sufficient to activate the chemogenetically activated protein in NKB-expressing neurons of the subject. In some embodiments, the chemogenetically activated protein is a modified human M3 muscarinic receptor (hM3Dq) and the agent is clozapine-N-oxide (CNO). Various chemogenetically activated proteins have been reported, including hM3Dq, an engineered version of a G-protein coupled receptor that is typically involved in regulation of insulin homeostasis. hM3Dq lacks constitutive activity but is selectively activated upon binding to CNO, an otherwise pharmacologically inert compound (see, e.g., Alexander, et al. “Remote control of neuronal activity in transgenic mice expressing evolved G protein-coupled receptors.” Neuron. 2009; 63 (1): 27-39, which is incorporated by reference herein). Further examples of chemogenetically activated proteins and activating chemical agents, such as, but not limited to, perlapine, deschloroclozapine, and compound 21, are generally known in the relevant art (see, e.g., Miura, et al., “Chemogenetics of cell surface receptors: beyond genetic and pharmacological approaches.” RSC Chem Biol. 2022; 3 (3): 269-287, which is incorporated by reference herein). In some embodiments, the chemogenetically activated protein and/or chemical agent is an alternate chemogenetically activated protein and/or chemical agent that is generally known in the art. In some embodiments, the vector is administered to the NKB-expressing neurons. In some embodiments, the vector is a viral vector. In some embodiments, the viral vector is a recombinant adeno-associated virus (rAAV) vector (e.g., an rAAV vector that is capable of entering neurons located in the NAc), or another suitable viral vector known in the art.

In some embodiments, the administration occurs via injection. In some embodiments, the administration occurs via intravenous injection, intraperitoneal injection, or intracranial injection.

In some embodiments, the method comprises electrically stimulating activity of the NKB-expressing neurons in the subject. Various techniques for electrically stimulating activity of select cell population in the brain are known in the art. For example, in some embodiments, the method comprises treating NKB-expressing neurons in the subject with deep brain stimulation (DBS) (see, e.g., Neumann, et al. “A practical guide to invasive neurophysiology in patients with deep brain stimulation.” Clin Neurophysiol. 2022 August; 140:171-180, which is incorporated by reference herein).

As used herein, the terms “administer,” “administering,” or “administration” refer to implanting, absorbing, ingesting, injecting, inhaling, or otherwise introducing an agent described herein, or a composition thereof (e.g., a pharmaceutical composition), in or on a subject. As used herein, the term “treatment.” “treat,” and “treating” refers to the application or administration of an agent described herein, or a composition thereof (e.g., a pharmaceutical composition), to a subject in need thereof for the purpose of reducing the severity of a disease (e.g., a drug addiction) in the subject. A “subject in need thereof” refers to an individual that has a disease, a symptom of the disease, or a predisposition toward the disease. A method for treating a disease may encompass administering to a subject an agent described herein, or a composition thereof (e.g., a pharmaceutical composition) with the intention to cure, heal, alleviate, relieve, alter, remedy, ameliorate, improve, or affect the disease, a symptom of the disease, or predisposition toward the disease in the subject. A method for treating a disease may encompass prophylaxis, wherein an agent is administered to the subject for the purpose of preventing development of the disease, for example, in a subject that is not known to have the disease, but may develop or be at risk of developing the disease in the future.

As used herein, a “therapeutically effective amount” or “effective amount” refers to the amount of an agent that is sufficient to elicit the desired biological response in the subject, for example, alleviating one or more symptoms of a disease (e.g., a drug addiction). A therapeutically effective amount may be an amount that is either administered to the subject alone or in combination with one or more other agents. Effective amounts vary, as recognized by those skilled in the art, depending on such factors as the desired biological endpoint, the pharmacokinetics of the administered agent, the particular condition or disease being treated, the severity of the condition or disease, the individual parameters of the subject, including age, physical condition, size, gender and weight, the duration of the treatment, the nature of any other concurrent therapy, the specific route of administration, and like factors that are within the knowledge and expertise of the health practitioner to determine. These factors are well known to those of ordinary skill in the art and can be addressed with no more than routine experimentation. It is generally preferred that a maximum dose of the individual agents described herein or any combinations thereof to be used is at most the highest dose that can be safely administered to the subject according to sound medical judgment. Preferably, an effective dose is lower than the highest dose that can be safely administered to the subject. It will be understood by those of ordinary skill in the art, however, that a subject or health practitioner may select a lower dose (e.g., the minimum effective dose) in order to mitigate any potential risks of treatment, such as side effects of the treatment.

In some embodiments, for an adult subject of normal weight, doses ranging from about 0.01 to 1000 mg/kg of an agent may be administered. In some embodiments, the dose is between 1 to 200 mg. The particular dosage regimen, i.e., the dose, timing, and repetition, will depend on the particular subject and that subject's medical history, as well as the properties of the agent (such as the pharmacokinetics of the agent) and other consideration well known in the art.

Treating a disease (e.g., a drug addiction) may include delaying the development or progression of the disease or reducing disease severity. Treating the disease does not necessarily require curative results. As used herein, “delaying” the development of a disease means to defer, hinder, slow, retard, stabilize, and/or postpone progression of the disease in a subject. This delay can be of varying lengths of time, depending on the history of the disease and/or individuals being treated. A method that delays the development of a disease, or delays the onset of the disease, is a method that reduces probability of developing one or more symptoms of the disease in a given time frame and/or reduces extent of the symptoms in a given time frame, as compared to the absence of such a method. Comparisons are typically based on clinical studies, using a number of subjects sufficient to give a statistically significant result.

The “development” or “progression” of a disease (e.g., a drug addiction) refers to initial manifestations and/or ensuing progression of the disease in a subject. Development of a disease can be detectable and assessed using standard clinical techniques as well known in the art. However, development also refers to progression that may be undetectable. For purpose of this disclosure, development or progression may refer to the development or progression of symptoms of a disease. The term “development” includes the occurrence, recurrence, and onset of a disease. As used herein “onset” or “occurrence” of a disease includes the initial onset of a disease, as well as recurrence/relapse of the disease (i.e., in a subject who has had the disease previously).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or a non-human animal. In some embodiments, the non-human animal is a mammal (e.g., rodent, e.g., mouse or rat), a primate (e.g., cynomolgus monkey or rhesus monkey), a commercially relevant mammal (e.g., cattle, pig, horse, sheep, goat, cat, or dog), or a bird (e.g., commercially relevant bird, such as chicken, duck, goose, or turkey). The non-human animal may be a male or female at any stage of development and may be a juvenile animal or an adult animal. The non-human animal may be a transgenic animal or genetically engineered animal.

In some embodiments, the subject is a companion animal (e.g., a pet or service animal). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include rodents (e.g., rats, mice, guinea pigs, and hamsters), rabbits, or non-human primates.

In some embodiments, the administration occurs via injection. In some embodiments, the administration occurs via intravenous injection, intraperitoneal injection, or intracranial injection. In some embodiments, the administration occurs more than once. In some embodiments, the administration occurs once per day, once per 2 days, once per 3 days, once per 3 days, once per 4 days, once per 5 days, once per 6 days, once per week, once per 2 weeks, once per 3 weeks, once per month, once per 2 months, once per 3 months, once per 4 months, once per 5 months, once per 6 months, once per 7 months, once per 8 months, once per 9 months, once per 10 months, once per 11 months or once per year.

In some embodiments the method described herein results in increased signaling from NKB-expressing neurons of the NAc to the lateral hypothalamus. In some embodiments, signaling from NKB-expressing neurons of the NAc to the lateral hypothalamus is increased by at least 5%, increased by at least 10%, increased by at least 15%, increased by at least 20%, increased by at least 25%, increased by at least 30%, increased by at least 35%, increased by at least 40%, increased by at least 45%, increased by at least 50%, increased by at least 60%, increased by at least 65%, increased by at least 70%, increased by at least 75%, increased by at least 80%, increased by at least 85%, increased by at least 90%, increased by at least 95%, increased by at least 2-fold, increased by at least 3-fold, increased by at least 4-fold, increased by at least 5-fold, increased by at least 5-fold, increased by at least 6-fold, increased by at least 7-fold, increased by at least 8-fold, increased by at least 9-fold, or increased by at least 10-fold.

In some embodiments, the method described herein results in decreased drug reward behavior in the subject. In some embodiments, drug reward behavior in the subject is decreased by at least 5%, decreased by at least 10%, decreased by at least 15%, decreased by at least 20%, decreased by at least 25%, decreased by at least 30%, decreased by at least 35%, decreased by at least 40%, decreased by at least 45%, decreased by at least 50%, decreased by at least 60%, decreased by at least 65%, decreased by at least 70%, decreased by at least 75%, decreased by at least 80%, decreased by at least 85%, decreased by at least 90%, decreased by at least 95%, decreased by at least 99%, or decreased by 100%.

In some embodiments, the drug addiction is a drug addiction in which NKB-expressing neuron activity in the subject is reduced. In some embodiments, the drug addiction is a drug addiction in which NKB-expressing neuron activity in the subject is reduced by at least 5%, decreased by at least 10%, decreased by at least 15%, decreased by at least 20%, decreased by at least 25%, decreased by at least 30%, decreased by at least 35%, decreased by at least 40%, decreased by at least 45%, decreased by at least 50%, decreased by at least 60%, decreased by at least 65%, decreased by at least 70%, decreased by at least 75%, decreased by at least 80%, decreased by at least 85%, decreased by at least 90%, decreased by at least 95%, decreased by at least 99%, or decreased by 100%. In some embodiments, the drug addiction is selected from nicotine addiction, cocaine addiction, opioid addiction, alcohol addiction, barbiturate addiction, and methamphetamine addiction, or a combination thereof. In some embodiments, the drug addiction is an addiction to another drug that is generally known in the art.

### Compositions

The present disclosure further provides compositions for use in treating drug addiction in a subject. In some embodiments, such a composition comprises an agent for activating neurokinin B (NKB)-expressing neurons in the subject. In some embodiments, the NKB-expressing neurons are dopamine 1 receptor (D1)-expressing neurons. In some embodiments, the NKB-expressing neurons are medium spiny neurons (MSNs). In some embodiments, the NKB-expressing neurons are located in the nucleus accumbens (NAc) of the subject.

In some embodiments, the agent is a small molecule, a hormone, a protein, a peptide, and aptamer, or a nucleic acid. In some embodiments, the agent activates a G-protein coupled receptor expressed in NKB-expressing neurons of the subject. In some embodiments the agent is an agonist of a G-protein coupled receptor expressed in NKB-expressing neurons of the subject. In some embodiments, the G-protein coupled receptor is Neurokinin 3 Receptor (NK3R), Thyrotropin Releasing Hormone Receptor (TRHR), or G-Protein Coupled Receptor 158 (GPR158). In some embodiments, the G-protein coupled receptor is NK3R and the agent is an agonist of NK3R. In some embodiments, the agonist of NK3R is senktide.

In some embodiments, the agent comprises a nucleic acid encoding an active form of a G-protein coupled receptor. In some embodiments, the agent comprises a nucleic acid encoding an active form of NK3R, TRHR, or GPR158. In some embodiments, the agent is a viral vector comprising a nucleic acid encoding an active form of a G-protein coupled receptor (e.g., an active form of NK3R. TRHR, or GPR158).

In some embodiments, the agent is a vector encoding an optogenetically activated protein, wherein laser stimulation activates the optogenetically activated protein, thereby activating NKB-expressing neurons of the subject. In some embodiments, the optogenetically activated protein is light-gated cation channel channelrhodopsin (ChR2) and the laser stimulation is blue light laser stimulation. In some embodiments, the optogenetically activated protein and/or laser stimulation are selected from another optogenetically activated protein and/or laser stimulation that is generally known in the art. In some embodiments, the vector is a viral vector, in some embodiments, the viral vector is a recombinant adeno associated virus (rAAV) vector (e.g., an rAAV vector that is capable of entering neurons located in the NAc), or another suitable viral vector known in the art.

In some embodiments, the agent comprises a vector encoding a chemogenetically activated protein, wherein administration of a chemical agent is sufficient to activate the chemogenetically activated protein, thereby activating NKB-expressing neurons of the subject. In some embodiments, the chemogenetically activated protein is a modified human M3 muscarinic receptor (hM3Dq) and the chemical agent is clozapine-N-oxide (CNO). In some embodiments, the chemogenetically activated protein and/or chemical agent are selected from another chemogenetically activated protein and/or chemical agent that is generally known in the art. In some embodiments, the vector is a viral vector, in some embodiments, the viral vector is a recombinant adeno associated virus (rAAV) vector (e.g., an rAAV vector that is capable of entering neurons located in the NAc), or another suitable viral vector known in the art.

In some embodiments, the composition further comprises a pharmaceutically acceptable excipient. “Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable excipient” may be a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each excipient must be “acceptable” in the sense of being compatible with the other ingredients of the formulation and not injurious to the tissue of the patient (e.g., physiologically compatible, sterile, physiologic pH, etc.). The term “excipient” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for administration to a subject, each unit containing a predetermined quantity of active material calculated to produce the desired therapeutic effect in association with the required diluent; i.e., excipient, or vehicle.

The formulation of the pharmaceutical composition may dependent upon the route of administration to a subject. Injectable preparations suitable for parenteral administration or intraperitoneal, intratumoral, peritumoral, intralesional or perilesional administration include, for example, sterile injectable aqueous or oleaginous suspensions and may be formulated according to the known art using suitable dispersing or wetting agents and suspending agents. The injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In some embodiments, compositions (e.g., pharmaceutical compositions administered to a subject) provided herein must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

In some embodiments, the composition is suitable for administration by injection. In some embodiments, the composition is suitable for administration by intravenous injection, intraperitoneal injection, or intracranial injection.

In some embodiments, the composition is administered to the subject more than once. In some embodiments, the composition is administered to the subject once per day, once per 2 days, once per 3 days, once per 3 days, once per 4 days, once per 5 days, once per 6 days, once per week, once per 2 weeks, once per 3 weeks, once per month, once per 2 months, once per 3 months, once per 4 months, once per 5 months, once per 6 months, once per 7 months, once per 8 months, once per 9 months, once per 10 months, once per 11 months or once per year.

In some embodiments, administration of the composition to a subject results in increased signaling from NKB-expressing neurons of the NAc of the subject to the lateral hypothalamus of the subject. In some embodiments, administration of the composition to a subject results in decreased drug reward behavior in the subject. In some embodiments, the subject is a human

In some embodiments, the drug addiction is a drug addiction in which NKB-expressing neuron activity in the subject is reduced. In some embodiments, the drug addiction is selected from nicotine addiction, cocaine addiction, opioid addiction, alcohol addiction, barbiturate addiction, and methamphetamine addiction, or a combination thereof.

### EXAMPLES

### Example 1—Neurokinin B-Expressing D1 Medium Spiny Neurons Negatively Regulate Cocaine Addiction

### Introduction

Drug addiction (also referred to as substance use disorder) is a chronic, relapsing disorder characterized by compulsive drug seeking and taking, resulting in the incapability of self-control and harmful consequences¹. As an important component of the basal ganglia and brain reward circuitry, the nucleus accumbens (NAc) plays a critical role in processing reward and motivation-related information²⁻⁷, and its role in drug addiction has been intensively studied for decades⁸⁻¹². Although these studies have provided important insights into the roles of NAc in regulating different aspects of addiction-related behavior, some important questions remain unanswered. For example, it has been shown that different subregions, such as the ‘core’ and the ‘shell,’^{13,14} and different inputs/outputs¹⁵⁻¹⁷ of the NAc are involved in different aspects of addiction-related behavior. However, it is unknown whether the functional diversity of NAc can be attributed to its different neuronal substrates, and it remains challenging to explain some discordant findings regarding the causal roles of dopamine 1 receptor (D1)-medium spiny neurons (MSNs) in regulating reward-related behaviors^{10,17,18}. This is at least partially due to the incomplete understanding of the neuronal composition within the NAc.

The conventional direct/indirect pathway model, which divides the striatal projection neurons into D1 and D2 MSN, has inspired the understanding of the cellular, circuitry, and functional organization of the striatum since it was proposed¹⁹⁻²⁴. However, this model largely ignored the intra-striatal heterogeneity, and thus cannot explain the substantial molecular, anatomic, and functional heterogeneity observed in the NAc^{18,25-28}. Consistent with this notion, recent single-cell RNA-sequencing (scRNA-seq) and MERFISH studies have revealed rich neuron subtypes beyond the D1/D2 MSN dichotomy model²⁹⁻³¹, suggesting that the molecularly defined D1/D2 MSN subtypes may play different roles during the addictive process. Furthermore, some recent studies have revealed functionally distinct subpopulations of striatal D1/D2 MSNs are involved in reinforcement learning and drug-related behavior^{18,28,32-38}. However, direct evidence of molecularly defined MSN subtypes beyond the pan-D1/D2 populations in regulating addiction-related behavior remains lacking.

The function of tachykinin 2 (Tac2)-expressing MSNs was therefore investigated in mouse models of cocaine addiction. Tac2-expressing MSNs (neurokinin B (NKB)-expressing MSNs in humans) are one of the 30 distinct D1 MSN subtypes located in the NAc dorsomedial shell³¹. By combining single-cell Ca²⁺ imaging and cell type-specific activity manipulation, an unexpected role was determined for Tac2⁺ neurons in negatively regulating cocaine reward and contingent drug-taking, without affecting the psychostimulatory effect of cocaine. These findings highlight the importance of using the cell-type specific approach in understanding drug addiction and highlight new opportunities for the treatment of drug addiction.

### Results

### Tac2-Expressing D1 MSNs Activity is Suppressed by Cocaine Administration

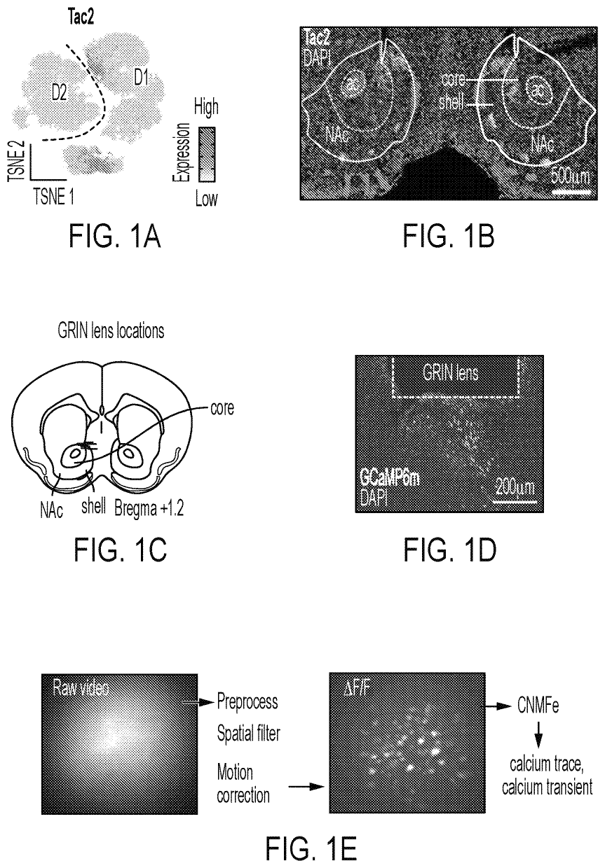

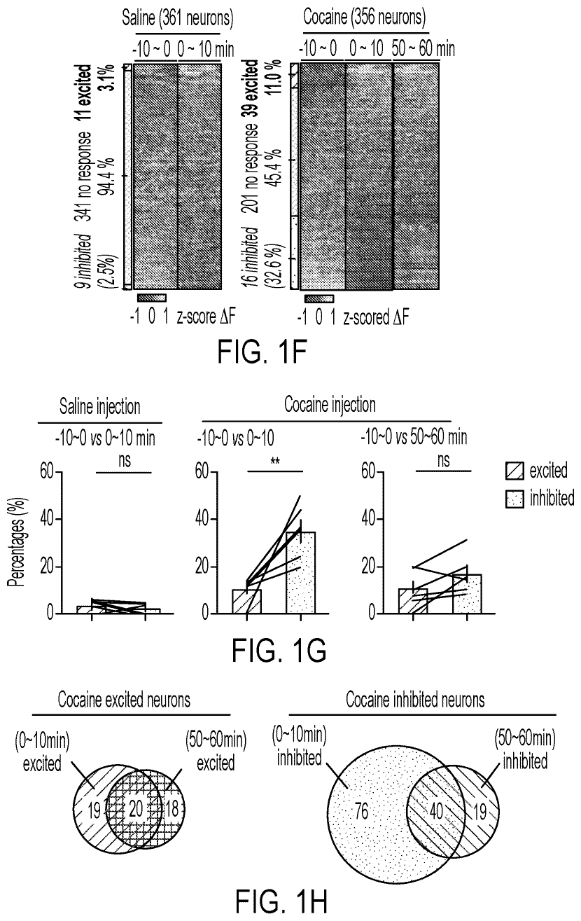

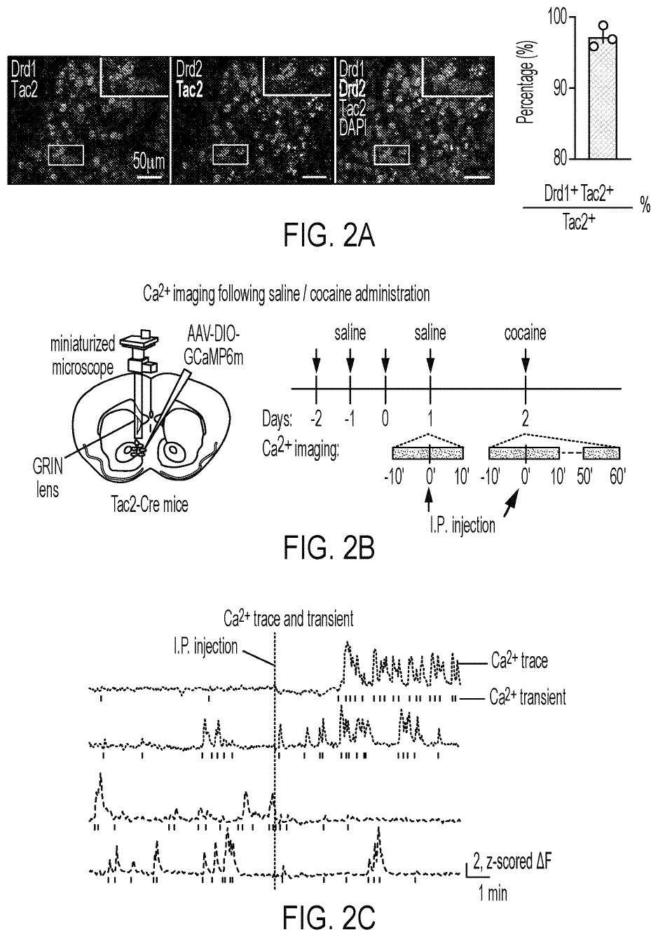

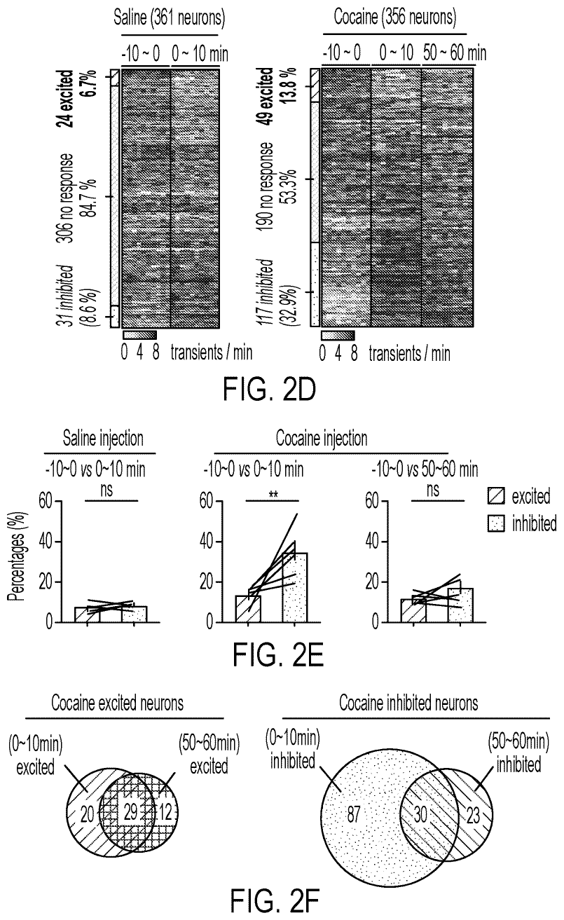

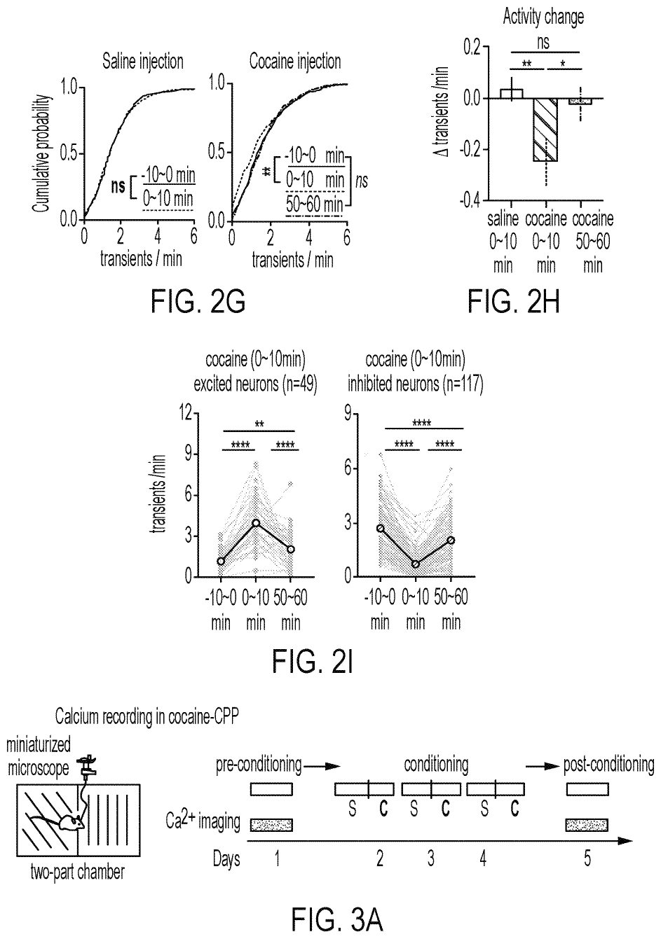

Single-cell RNA sequencing (scRNA-seq) of mouse NAc neurons revealed that tachykinin 2 (Tac2) is selectively expressed in one of the 30 D1 MSN subtypes that were recently identified and accounts for ˜5% of the total NAc D1 MSNs³¹ (FIG. 1A). Similarly, a recent study in primate has identified an equivalent novel D1 archetype in the primate striatum³⁹. In situ RNA hybridization revealed that Tac2-expressing neurons are located in the dorsomedial part of the NAc shell and core (FIG. 1B), a region associated with drug reward processing⁴⁰. Multi-color RNA hybridization further confirmed that Tac2 is predominantly expressed in a subpopulation of D1 MSNs, with little overlap with D2 MSNs (FIG. 2A). The cellular and spatial properties of Tac2⁺ neurons suggest that they may be involved in drug reward-related behaviors.

To explore a potential role for Tac2⁺ neurons in drug-related behavior, Tac2 neuronal activity was monitored using single-cell calcium imaging upon psychostimulant drug (cocaine) treatment. To this end, GCaMP6m, which reports calcium fluctuations and serves as a proxy for neuronal activity, was virally delivered into the NAc of Tac2-Cre mice using known techniques⁴¹, and the fluorescence signal was monitored by a skull-attached miniaturized microscope through a gradient-index (GRIN) lens implanted above the virus injection site (FIG. 2B, FIGS. 1C-1D). Cocaine levels in both brain and plasma peak within 10 minutes after administration, and decay one hour later^{42,43}, therefore calcium signal was recorded in one 10-minute session during pre-injection (baseline: −10-0 minutes) and two 10-minute sessions (initial phase: 0-10 minutes, and decay phase: 50-60 minutes) during post-injection (i.p. injection) of cocaine (15 mg/kg) (FIG. 2B). After extracting calcium signal traces and corresponding calcium signal transients of individual neurons from raw imaging video (FIG. 2C, FIG. 1E), the Tac2 neuronal response to saline and cocaine administration was analyzed. The calcium transient frequency from 6 mice (saline: 361 neurons, cocaine 356 neurons) were sorted and presented as heatmaps (FIG. 2D). Upon saline injection, only a small proportion of Tac2⁺ neurons showed significant changes in their activity (24/361 or 6.7% neuron increased activity, 31/361 or 8.6% neurons decreased activity) (FIG. 2D). In contrast, cocaine injection significantly increased the number of responsive Tac2⁺ neurons, with 13.8% (49/356) and 32.9% (117/356) of recorded neurons showing increased and decreased activity, respectively, in the initial phase (0-10 minutes). The numbers of cocaine-responsive neurons declined in the decay phase (50-60 minutes) with 11.5% (41/356) and 14.9% (53/356) of recorded neurons showing increased and decreased activity, respectively (FIG. 2E). Among the cocaine initial phase responsive neurons, 59.2% (29/49) of the cocaine excited neurons and 25.6% (30/117) of the cocaine inhibited neurons are respectively excited and inhibited in the decay phase (FIG. 2F), indicating that Tac2⁺ neurons exhibit dynamic response to cocaine administration. Interestingly, 2.4-fold more Tac2⁺ neurons are inhibited by cocaine when compared to those excited by cocaine in the initial phase (FIG. 2E), and an overall decrease of Tac2 neuronal activity following cocaine injection was observed, which was not the case in the saline treatment (FIG. 2G-2H). The neuronal activity of cocaine excited/inhibited Tac2⁺ neurons in the initial phase were substantially altered following cocaine injection and later partially recovered in the decay phase (FIG. 2I). To further validate these findings, calcium activity traces were analyzed before and after saline or cocaine injection. 3.0-fold more cocaine inhibited neurons than cocaine excited neurons in the initial phase (FIGS. 1F-1H). Collectively, these results demonstrate that acute cocaine administration modulates the activity of a significant percentage of Tac2⁺ neurons, with the majority of them showing decreased neuronal activity.

### Cocaine Place Conditioning Modulates the Activity of Tac2+ Neurons

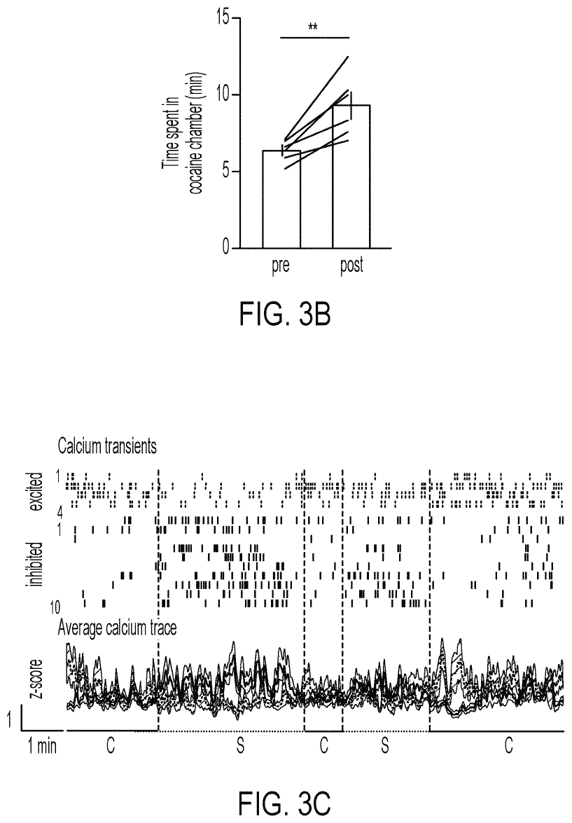

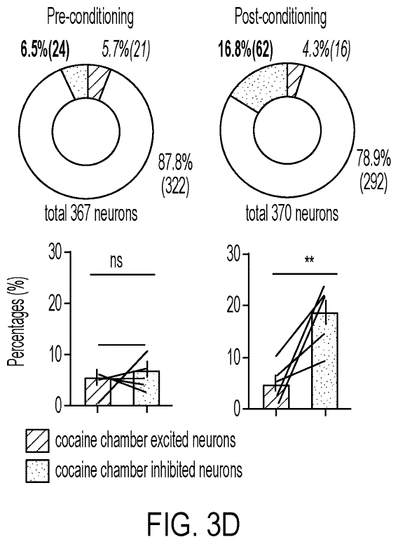

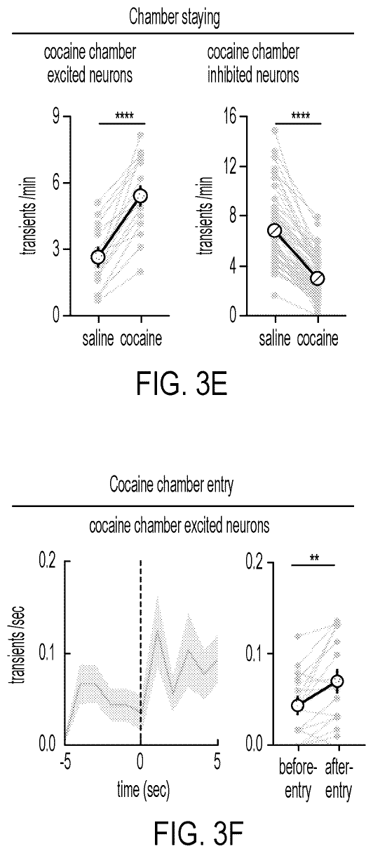

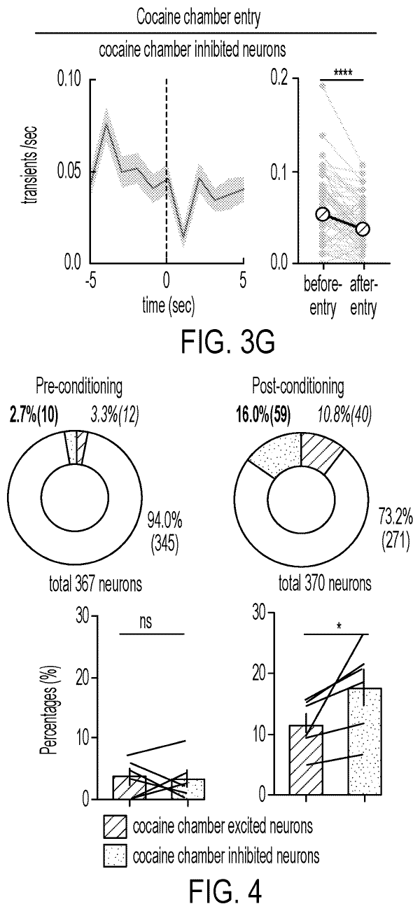

Next, the dynamics of Tac2 neuronal activity was analyzed during cocaine reward-contextual association. To this end, single-cell calcium imaging was applied to a cocaine-conditioned place preference (CPP) test (FIG. 3A). In the pre-conditioning session, mice were allowed to freely explore the two chambers with differential contextual patterns while calcium signal being recorded. In the conditioning sessions, mice were given alternate saline and cocaine (15 mg/kg) injections (6 hours apart) and were confined to a specific chamber paired with saline or cocaine. In the post-conditioning session, mice were again allowed to freely explore the two chambers while calcium signal being recorded (FIG. 3A). As expected, mice exhibited a significant preference to the cocaine chamber after conditioning (FIG. 3B), indicating the establishment of a cocaine-related contextual memory. After extracting the calcium signal traces, and corresponding calcium signal transients of individual neurons in pre-conditioning and post-conditioning sessions, signal traces or transient frequencies were compared during the periods when mice are in saline-coupled chamber or cocaine-coupled chamber (staying time>5 seconds). A neuron was identified as cocaine-associated context encoding (CACE) neuron if the averaged calcium transient frequencies were significantly altered when mice stayed in the two different chambers. Interestingly, it was observed that CACE neurons exhibit either higher or lower calcium signal in the cocaine chamber when compared to that in the saline chamber (FIGS. 3C, 3E). In the pre-conditioning session, a comparable proportion of recorded neurons that showed higher (21 out of the 367, or 5.7%) or lower (24 out of the 367, or 6.5%) activity in the cocaine chamber (FIG. 3D), suggesting that Tac2⁺ neurons do not distinguish the two chambers before cocaine conditioning. However, after cocaine conditioning, the Tac2⁺ neurons that showed reduced activity in the cocaine chamber (62 out of 370, or 16.8%) were significantly increased, while those that showed elevated activity in the cocaine chamber were slightly decreased (16 out of 370, or 4.3%) (FIG. 3D). To further validate these findings, calcium activity traces were analyzed when mice stayed in the saline chamber or cocaine chamber. In the pre-conditioning session, a small and comparable proportion of neurons altered their neuronal activity when stayed in the cocaine chamber (12 out of 367, or 3.3% excited, 10 out of 367, or 2.7% inhibited). In the post-conditioning session, a significant increase in the CACE neurons with a larger proportion of cocaine chamber inhibited neurons was observed (40 out of 370, or 10.8% excited, 59 out of 370, or 16.0% inhibited) (FIG. 4A). Interestingly, it was observed that the CACE neurons emerging after cocaine conditioning rapidly changed their activity upon mice entering the other chamber, calcium traces and corresponding transients of CACE neurons from a representative mouse clearly showed the two groups of neurons with opposite response patterns when the animal was staying in the two chambers (FIG. 3C), suggesting these neurons could distinguish the environment associated with saline and cocaine after conditioning. Moreover, by focusing on the short period around the mice entering the cocaine chamber (−5 seconds to 5 seconds, regarding the entering as 0 second), it was determined that the neuronal activity of the cocaine chamber excited neurons (neurons with higher calcium signal in cocaine chamber) increased rapidly when mice entered the cocaine chamber, while the cocaine chamber inhibited neurons (neurons with higher calcium signal in cocaine chamber) decreased their activity when mice entered the cocaine chamber (FIGS. 3F-3G). This result suggested that cocaine context-responsive neurons might also involve in the decision making of chamber entries. Overall, these results suggest that cocaine conditioning modulates Tac2 neuronal activities that related to cocaine-reward contextual associations.

### NAc Tac2⁺ Neurons Bidirectionally Regulate Cocaine-Induced Place Preference

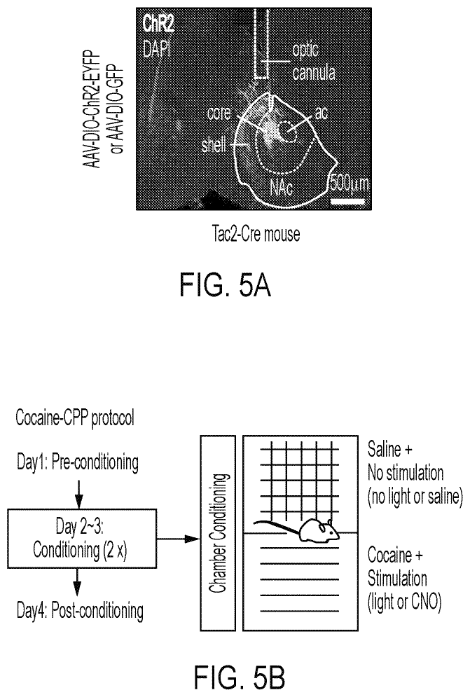

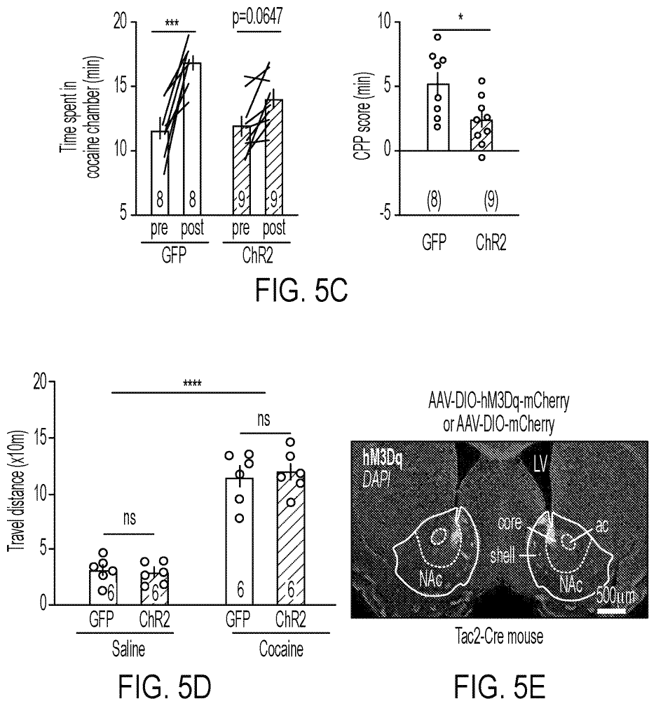

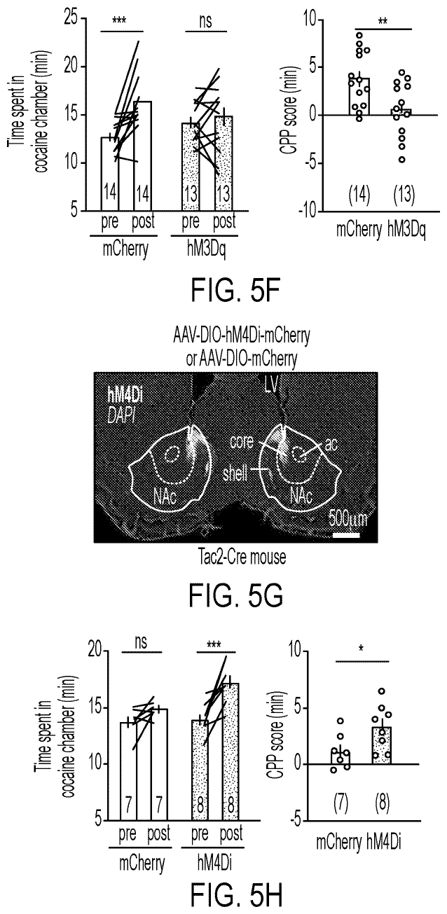

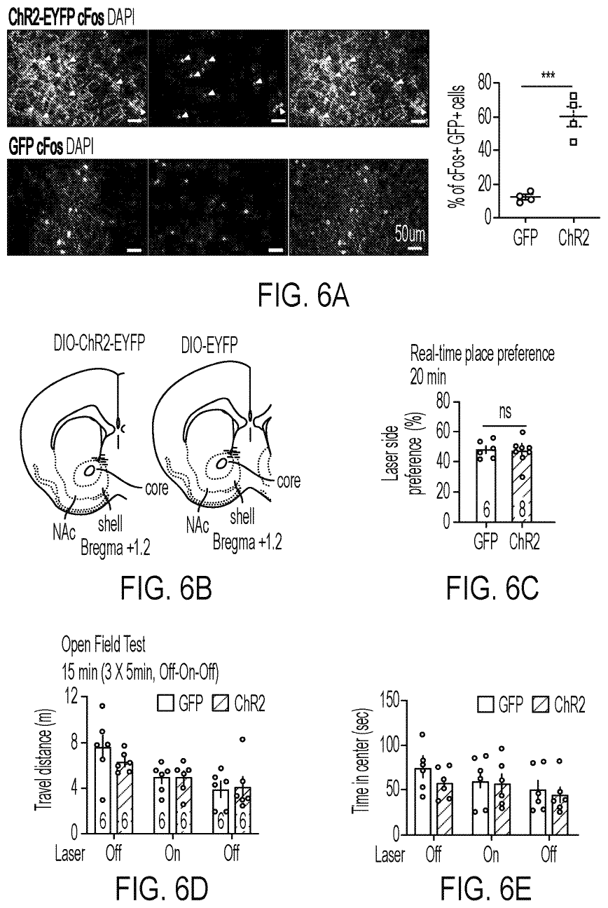

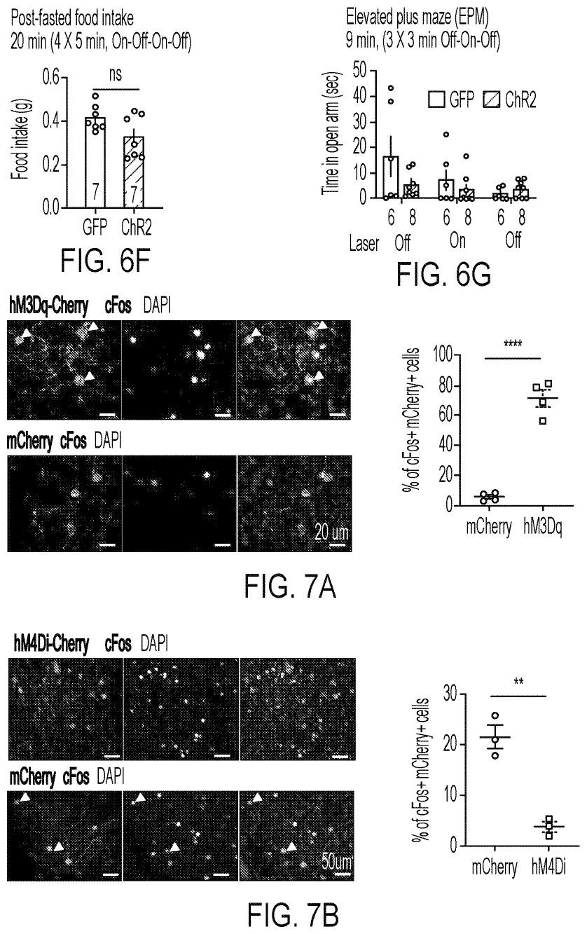

Since both acute cocaine administration (FIG. 2) and cocaine place conditioning (FIG. 3) could modulate the activity of Tac2⁺ neurons, it was hypothesized that Tac2⁺ neurons are involved in regulating cocaine-related behaviors. To test this hypothesis, AAV vectors expressing light-gated cation channel channelrhodopsin (ChR2) or GFP were injected into the NAc of two cohorts of Tac2-Cre mice, and optical cannulas were implanted above the viral injection sites for light delivery (FIG. 5A). After confirming the efficacy of optogenetic activation of Tac2⁺ neurons with cFos induction (FIG. 6A) and post histological verification of optic cannulas locations (FIG. 6B), it was examined whether Tac2 neuronal activation affects cocaine reward in a conditioned place preference (CPP) model. Specifically, in the pre-conditioning session, mice were allowed to freely explore two interconnected chambers with different tactile cues to establish a baseline preference. Then, in the following conditioning session, mice were first conditioned with saline and no laser stimulation in one chamber for 30 minutes, and then conditioned with cocaine (15 mg/kg i.p.) and laser stimulation in the other chamber for 30 minutes (FIG. 5B). In the post-conditioning session, the preference of the mice to the two chambers was tested again. Cocaine conditioning significantly increased the preference of the GFP-expressing mice to the cocaine/laser chamber. However, this preference was largely attenuated by Tac2 neuronal activation in the ChR2-expressing mice (FIG. 5C), suggesting that activation of Tac2⁺ neurons attenuated the formation of cocaine CPP. Recent studies have revealed that some D1 MSNs in the NAc encode an aversive signal¹⁸, raising the possibility that activating Tac2⁺ neurons may confer such negative valence, thus attenuating the rewarding effect of cocaine in the CPP tests. However, activation of Tac2⁺ neurons had no significant effect on real-time place preference (FIG. 6C), suggesting that the function of Tac2⁺ neurons in regulating cocaine CPP could not be explained by adding an extra reward/aversive effect to the drug. Notably, manipulation of Tac2 neuronal activity affected neither basal locomotion in open arena (FIG. 6D), time spent in the center area of open field arena (FIG. 6E), nor cocaine-induced hyperlocomotion (FIG. 5D). In addition, although previous studies have established a role for NAc D1 MSN in feeding⁴⁴ and anxiety⁴⁵. Tac2 neuronal activation affected neither food intake nor elevated plus maze test (FIGS. 6F-6G), which supports the existence of functionally distinct D1 MSN subtypes.

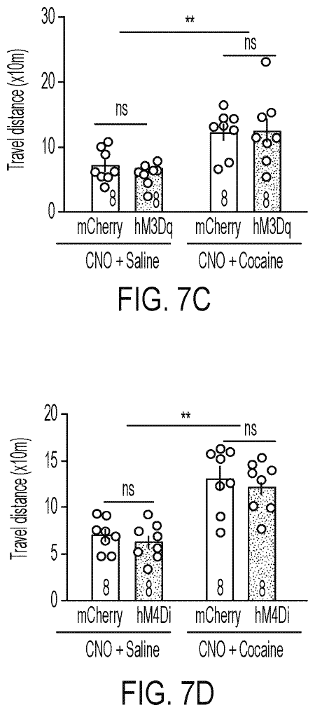

To further confirm the above observations, another set of cocaine-CPP experiments was performed using chemogenetic tools. To this end, a Cre-dependent AAV encoding a modified human M3 muscarinic receptor (hM3Dq) was introduced into NAc of Tac2-Cre mice (FIG. 5E). Administration of clozapine-N-oxide (CNO, 2 mg/kg, i.p.) resulted in cFos induction in the hM3Dq-expressing neurons, but not in the mCherry-expressing neurons (FIG. 7A), confirming the efficacy and specificity of chemogenetic activation of Tac2⁺ neurons. The cocaine-CPP test was then repeated, in which mice were conditioned with cocaine plus CNO in one chamber, while with saline in the other chamber. Similar to the optogenetic activation result, chemogenetic activation of the Tac2⁺ neurons completely abolished cocaine-induced CPP (FIG. 5F). Previous studies have demonstrated that optogenetic activation of pan-D1 MSNs in NAc promoted cocaine CPP¹⁰. Surprisingly, here activation of Tac2⁺ neurons, a subtype of D1 MSN, attenuated cocaine reward behavior. To further confirm these results, the effect of Tac2 neuronal inhibition on cocaine CPP was examined. To this end, chemogenetic inhibitory vector AAV-DIO-hM4Di-mCherry was injected into NAc of Tac2-Cre mice (FIG. 5G), and it was observed that CNO treatment (5 mg/kg, i.p.) significantly decreased cFos induction in the hM4Di-expressing neurons following cocaine exposure (FIG. 7B), indicating successful inhibition of the neuronal activity of the Tac2⁺ neurons. To test the effect of Tac2 neuronal inhibition on cocaine-CPP, a subthreshold cocaine CPP paradigm was applied, in which mice were conditioned with a lower dose of cocaine (10 mg/kg i.p.) in a shorter conditioning session (15-minutes per session)^{10,46}. CNO treatment (5 mg/kg, i.p.) resulted in a significantly higher cocaine CPP in the hM4Di-expressing mice when compared to mCherry-expressing mice (FIG. 5H), suggesting that suppression of Tac2 neuronal activity enhanced the reward effect of cocaine. Similar to the optogenetic activation of the Tac2⁺ neurons, chemogenetic-mediated bidirectional manipulation of Tac2⁺ neurons affected neither basal locomotion nor cocaine-induced hyperlocomotion (FIGS. 7C-7D), further support the functional specificity of the Tac2 expressing D1 MSN subtype in regulating different cocaine-related behaviors. Collectively, these results demonstrate that Tac2-expressing D1 MSNs play critical roles in regulating cocaine reward behaviors.

### NAc Tac2⁺ Neurons Regulate Cocaine Intravenous Self-Administration Behavior

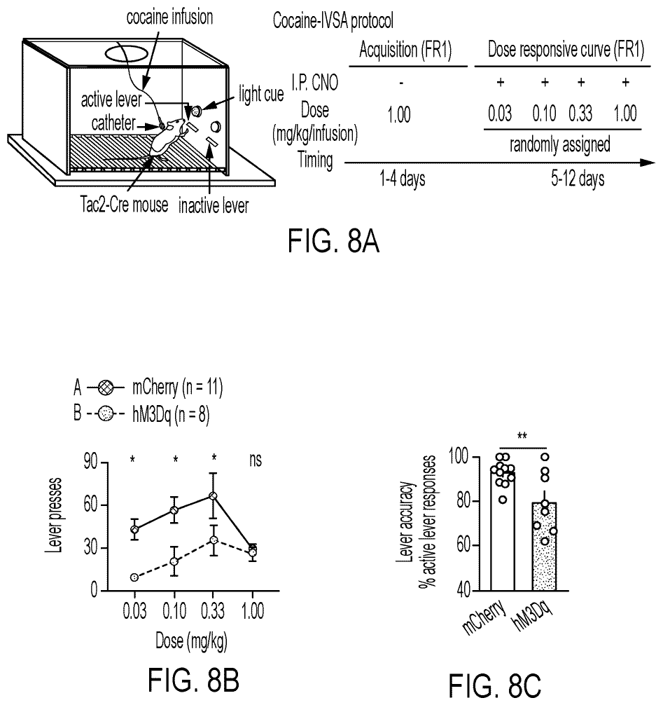

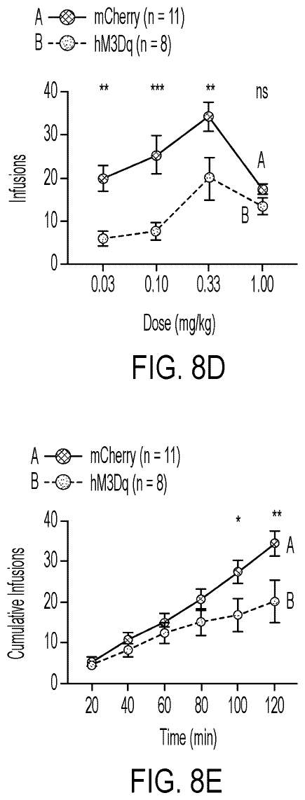

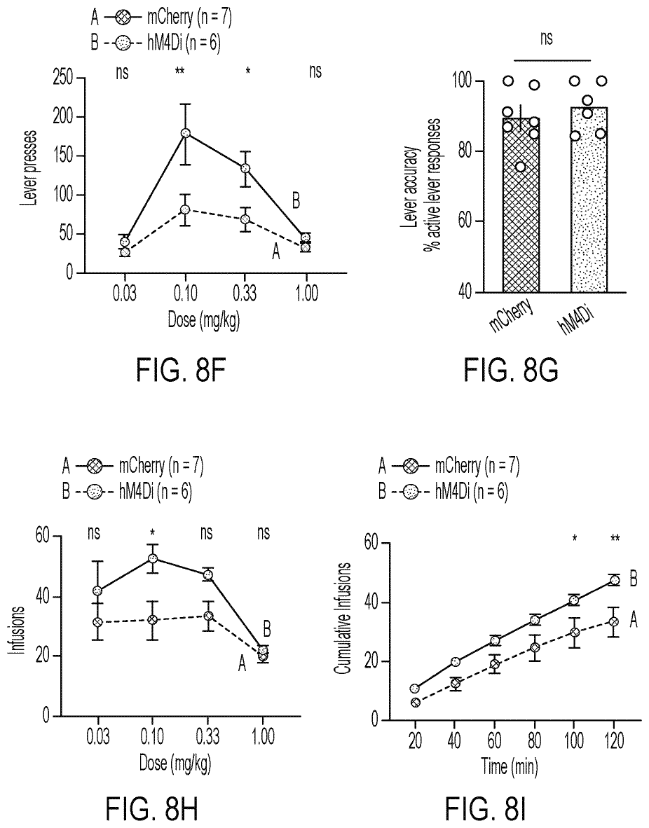

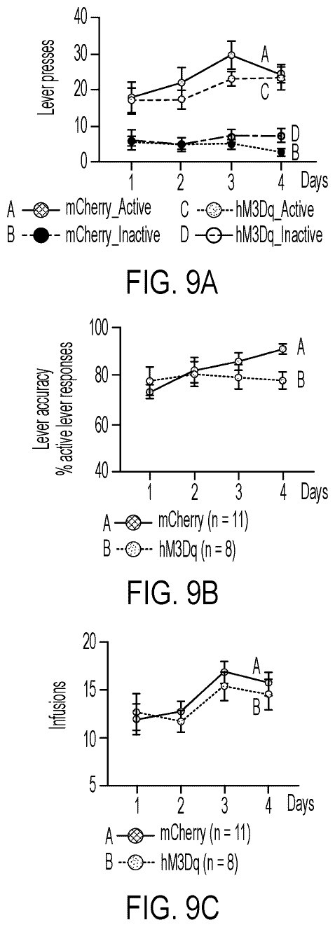

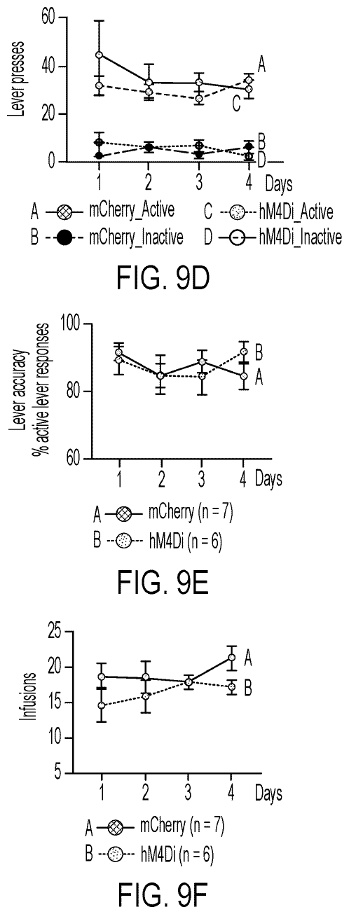

Having established a critical role of the NAc Tac2⁺ neurons in regulating cocaine reward behavior, the function of Tac2⁺ neurons in cocaine intravenous self-administration (cocaine-IVSA), a clinically relevant drug addiction model, was further examined. In this model, mice with indwelling jugular catheters were trained to press the active lever to self-administrate cocaine (1 mg/kg/infusion) in the operant chamber, while pressing the inactive lever would yield no outcome (FIG. 8A). To evaluate the effect of Tac2 neuronal activation, the drug-taking behavior was compared between hM3Dq- and mCherry-expressing mice (different cohorts from the ones used in the above cocaine-CPP test). Following 4-days training, mice showed significantly higher numbers of active lever presses than that of inactive lever presses (FIGS. 9A-9B), suggesting successful acquisition of cocaine self-administration behavior. Then the cocaine taking was compared between the two groups under different cocaine doses (0.03, 0.1, 0.3, 1 mg/kg/infusion) following CNO administration (FIG. 8B). The hM3Dq group showed significantly fewer lever presses (FIG. 8B), reduced lever accuracy (FIG. 8C), and fewer cocaine infusions (FIGS. 8D-8E), compared to the control mCherry-expressing group, suggesting that Tac2 neuronal activation suppressed the cocaine self-administration behavior. To evaluate the effect of Tac2 neuronal inhibition, the drug-taking behavior was compared between hM4Di- and mCherry-expressing mice (the same cohort of mice used in FIG. 5 were used, but the two experiments were performed with more than 1 week of gap time to avoid potential CNO-induced changes) using the same cocaine IVSA paradigm. After acquiring stable self-administration behavior (FIGS. 9C-9D), mice were tested under different cocaine doses following CNO treatment. CNO treatment resulted in a significantly higher number of lever presses (FIG. 8F), comparable lever accuracy (FIG. 8G), and significantly lower number of cocaine infusions (FIGS. 8H-81) in hM4Di-expressing mice, as compared to the control mCherry-expressing group, suggesting that Tac2 neuronal inhibition promoted cocaine self-administration. Collectively, the bidirectional manipulation of Tac2⁺ neurons indicates that Tac2 neuronal activity negatively regulates the contingent cocaine-taking behavior.

### The NAc to LH Projection of Tac2+D1 MSNs Regulates Cocaine Reward Memory

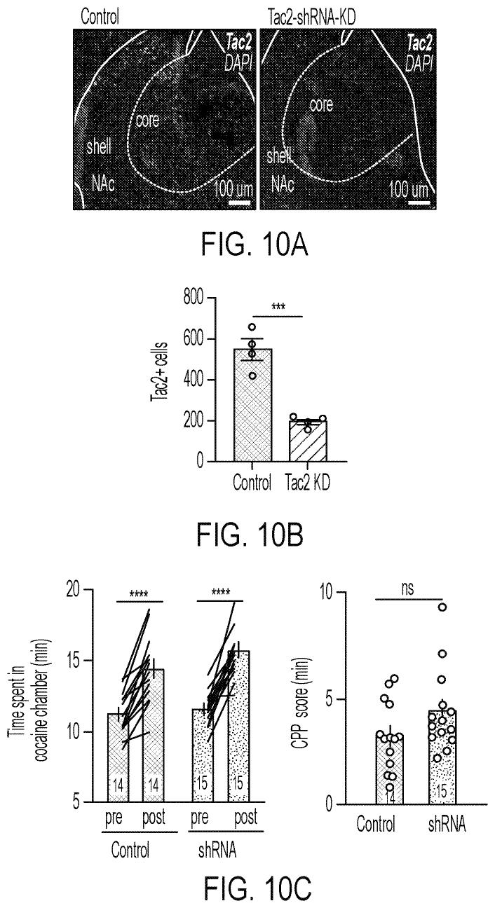

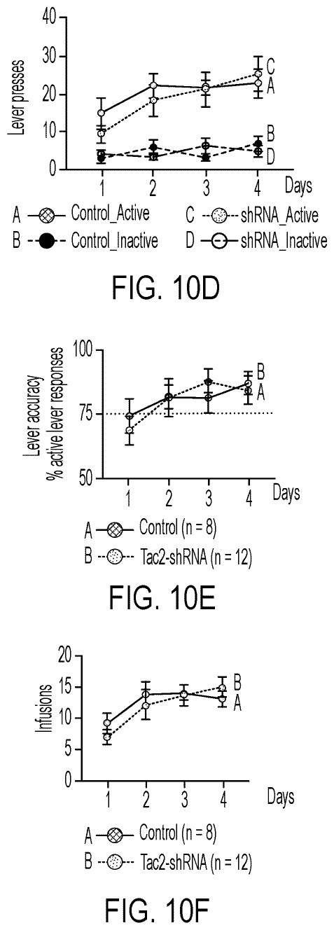

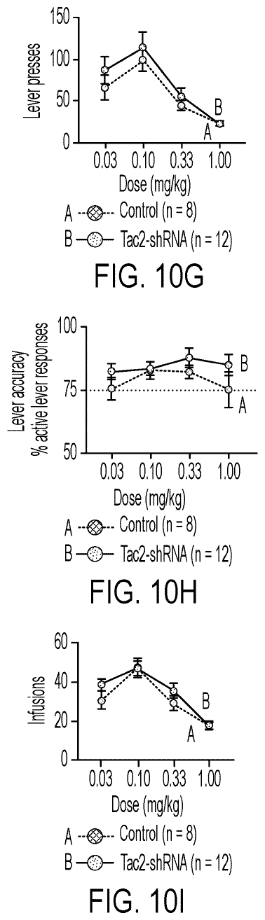

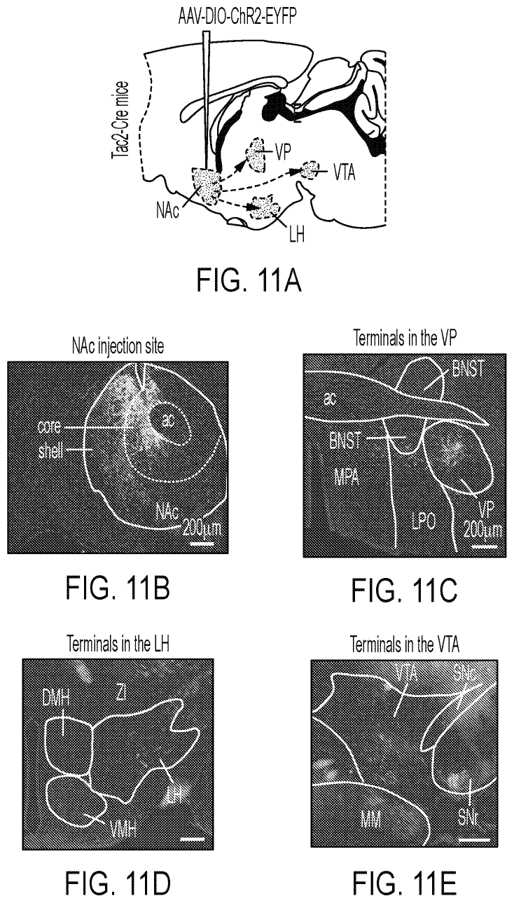

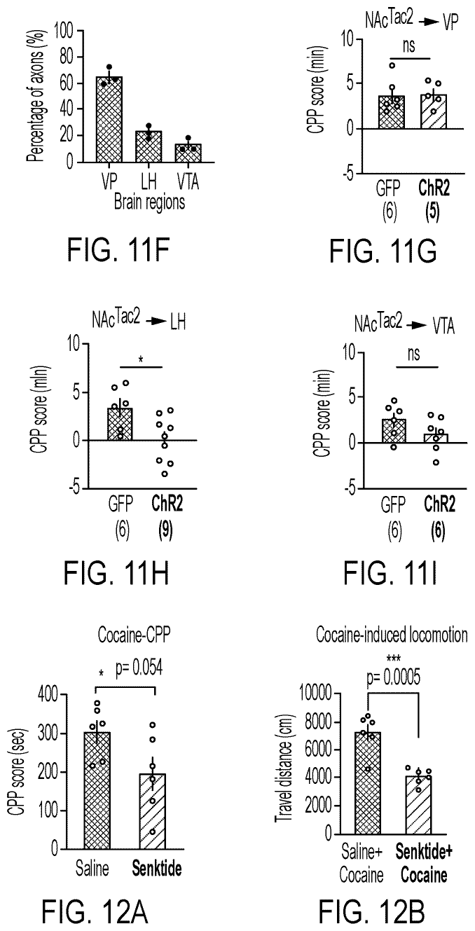

Using calcium recording and neuronal activity manipulation, a causal relationship has been established between Tac2 neuronal activity and cocaine-associated behaviors (CPP and IVSA). It was then explored how Tac2⁺ neurons might execute their functions. Given that the Tac2 gene encodes the neuropeptide neurokinin B (NKB), which has been implicated in various neurological processes, including social stress⁴⁷ and fear memory⁴⁸, it was tested whether the Tac2 encoded neuropeptide is involved. To this end, a genetic approach was employed to specifically knock-down Tac2 mRNA in the NAc region and then cocaine-related behaviors were tested. An AAV construct expressing a previously tested Tac2-shRNA⁴⁷ was packaged and bilaterally injected into the NAc region of wild-type mice. After confirming knockdown efficiency (FIGS. 10A-10B), mice were subjected to cocaine-related behavior tests. Tac2 knockdown neither affected cocaine-induced place preference (FIG. 10C), nor contingent cocaine taking (FIGS. 10D-10I). It was then investigated whether Tac2⁺ cells innervate downstream neurons through direct neuronal connection. To this end, ChR2-mediated antegrade tracing was performed (FIGS. 11A-11B), which revealed multiple projection targets of Tac2⁺ neurons, including the ventral pallidum (VP), the lateral hypothalamus (LH), and the ventral tegmental area (VTA) (FIGS. 11C-11F). To further determine which of these projections are involved in regulating cocaine reward behavior, AAV-DIO-ChR2 was injected to NAc, and optic cannulas were implanted into VP, LH, and VTA, respectively, for selective optogenetic activation of each of the projections upon behavioral tests. Optogenetic activation of the NAc to LH projection, but not the NAc to VP or NAc to VTA projection, significantly reduced the cocaine-CPP (FIG. 11D). These results suggest that NAc Tac2⁺ neurons regulate cocaine reward memory mainly through their projection to LH.

### Methods

### Mice

All experiments were conducted in accordance with the National Institute of Health Guide for Care and Use of Laboratory Animals and approved by the Institutional Animal Care and Use Committee (IACUC) of Boston Children's Hospital and Harvard Medical School. The Tac2-Cre knock-in mouse line was a gift from Dr. Qiufu Ma at Dana-Farber Cancer Institute and Harvard Medical School. The Tac2-Cre line was backcrossed to the C57BL/6N background. Mice were group-housed under controlled temperature (22-25° C.) conditions in a 12-hour light-dark cycle (light time, 7:00 to 19:00) with ad libitum chow food (4% fat SPF rodent feed) and water. All experiments were performed using male adult mice (8-16 weeks), and the behavioral experiments were all conducted during the light cycle.

### AAV Vectors

The following AAV vectors (with a titer of >10¹²) were purchased from UNC Vector Core: AAV5-EF1a-DIO-hChR2 (H134R)-EYFP, AAV5-EF1a-DIO-EYFP, AAV-DJ-EF1a-DIO-GCaMP6m. The following AAV vectors were obtained from Addgene: AAV5-hSyn-DIO-hM3D (Gq)-mCherry (#44361), AAV5-hSyn-DIO-hM4D(Gi)-mCherry (#44362), AAV5-hSyn-DIO-mCherry (#50459).

Tac2 shRNA Knock-Down

Small hairpin RNA (shRNA) for mouse Tac2 gene (NM_009312.2) were selected based on previous studies⁵⁰. The oligonucleotides encoding Tac2 shRNA were as follows: 5′-gatccgCCGCCTCAACCCCATAGCAATTAgaagcttgTAATTGCTATGGGGTTGAGGCttttttt-3′ (SEQ ID NO: 3) and 3′-gcGGCGGAGTTGGGGTATCGTTAATcttcgaacATTAACGATACCCCAACTCCGaaaaaaagat c-5′ (SEQ ID NO: 4). The oligonucleotides were cloned into the AAV vector backbone AAV-shRNA-Ctrl (Addgene, #85741), and both AAV-Tac2-shRNA and AAV-shRNA-Ctrl constructs were packaged by the Viral Core of Boston Children's Hospital. The AAV viruses were injected into NAc regions and knock-down efficiency were evaluated by Tac2 RNA FISH.

### Fluorescence In Situ Hybridization (FISH) and Immunofluorescence (IF) Staining

Mice were transcardially perfused with PBS followed by 4% paraformaldehyde. Brains were then placed in a 30% sucrose solution for 2 days. The brains were frozen in Optimal Cutting Temperature (OCT) embedding media and 16 μm (for FISH) or 40 μm (for IF) coronal sections were cut with a vibratome (Leica, no. CM3050 S). For FISH experiments, the slices were mounted on SuperFrost Plus slides, and air dried. The multi-color FISH experiments were performed following the instructions of RNAscope Fluorescent Multiplex Assay (ACD Bioscience). For IF, cryostat sections were collected and incubated overnight with blocking solution (1×PBS containing 5% goat serum, 2.5% BSA, and 0.1% Triton X-100), and then treated with the following primary antibodies, diluted with blocking solution, for 1 day at 4° C.: rabbit anti-cFos (1:2000, Synaptic systems, #226003), chicken anti-GFP (1:2000, Aves Labs, no. GFP-1010), and chicken anti-mCherry (1:2000, Novus Biologicals, no. NBP2-25158). Samples were then washed three times with washing buffer (1×PBS containing 0.1% Triton X-100) and incubated with the Alexa Fluor conjugated secondary antibodies for 2 h at room temperature. The sections were mounted and imaged using a Zeiss LSM800 confocal microscope or an Olympus VS120 Slide Scanning System.

### Image Processing and Quantification

NAc Tac2⁺ axonal projections were quantified by measuring the fluorescence signal intensity of targeted regions. Serial 40 μm coronal sections (spaced 120 μm apart) from Tac2-Cre::EF1a-DIO-hChR2 (H134R)-EYFP expressing mice were collected and imaged by VS120 Slide Scanning System (Olympus). Images collected were firstly pre-processed using OlyVIA software (Olympus) and then imported into Fiji software with background subtracted, brightness and contrast adjusted. Regions of interest were selected based on boundaries of brain regions defined by the mouse brain atlas⁵². Fluorescence signal intensities were measured using Fiji software.

### Stereotaxic Brain Surgeries

The AAV vectors were injected through a pulled-glass pipette and the nanoliter injector (Nanoject III, Drummond Scientific-3-000-207). The injection was performed using a small-animal stereotaxic instrument (David Kopf Instruments, model 940) under general anesthesia by isoflurane (0.8 liter/min, isoflurane concentration 1.5%) in oxygen. A feedback heater was used to keep mice warm during surgeries. Mice were allowed to recover in a warm blanket before they were transferred to housing cages for 2-4 weeks before the behavioral evaluation was performed. For chemogenetic experiments, 0.1˜0.15 μL of AAV vector was bilaterally delivered into target regions. For optogenetics experiments, following viral injection, the fiber optic cannulas (200 μm in diameter, Inper Inc. China) were implanted 0.3 mm above the viral injection site and were secured with dental cement (Parkell, #S380). The coordinates of viral injection sites are based on previous studies⁵² as follow: NAc (+1.2 mm AP, ±0.6 mm ML, −4.5 mm DV), VP (+0.1 mm AP, ±1.4 mm ML, −5.0 mm DV), LH (−1.3 mm AP, ±1.1 mm ML, −5.0 mm DV), and VTA (−3.2 mm AP, ±0.6 mm ML, −4.8 mm DV). For the calcium recording experiments, 0.3˜0.4 μL of viruses were injected into NAc region, 4˜5 weeks later, a gradient refractive index (GRIN) lens (0.6 mm diameter, 7 mm length, Inscopix) was implanted 0.3 mm above the viral injection site and was secured with dental cement. One week later, a baseplate that holds the miniature microscope (nVoke 1.0, Inscopix) was attached to the lens.

### Calcium Activity Imaging by a Miniature Endomicroscope

Two weeks after GRIN lens implantation surgery, a baseplate was implanted to mount the miniature microscope and to check the fluorescence signal. Freely moving in vivo imaging can be performed after the nVoke microscope (Inscopix) is attached to the fixed baseplate. All recordings were made at 10 frames per second, LED intensities were 0.6˜1.2 mW, and gain (1.5×) was used. The calcium traces were extracted using Inscopix Data Processing Software (version 1.6, Inscopix). The raw recording videos were cropped, spatially down-sampled (2×), spatially filtered (cut-offs: low 0.005 pixel⁻¹, high 0.500 pixel⁻¹), and motion-corrected (using a reference frame from the input movie). The spatial location of individual neurons and their associated calcium activity signals were identified using the extended constrained non-negative matrix factorization (CNMF-E) CNMF-c algorithm^{53,54} and outputted temporal traces as dF over noise. The identified neurons were manually checked to exclude overlapping neurons, blood vessels, or fluorescent debris moving in and out of focus. The calcium signal traces were further deconvolved using OASIS (online active set method to infer spikes) methods to estimate underlying calcium signal transients. The data matrix containing calcium signal traces and calcium signal transients were subjected to custom MATLAB code for further analysis.

### Calcium Imaging During Acute Saline/Cocaine Administration

Mice were tested in the homecages and were pre-habituated in the test room and they received 3-day saline injection to reduce stress. On day 1, mice were attached with nVoke microscope 30 minutes before experiment. Prior to injection, 10 minute baseline was recorded, then mice were intraperitoneally injected with saline, and a 10 minute recording was performed after the injection. On day 2, mice were again attached with n Voke microscope, and recorded for 10 minute baseline. Then mice were injected with cocaine (15 mg/kg) intraperitoneally. The post-injection initial phase (0-10 minutes) and decay phase (50-60 minutes) were recorded. Two data sets of neuronal activity were used: calcium signal traces over time or calcium signal transients over time, to analyze treatment (saline or cocaine injection) responsive neurons (excited or inhibited by treatment), 10-minute windows of pre- and post-injection were compared. For saline injection responsive neurons, saline (−10-0 minutes) and saline (0-10 minutes) responses of the two groups were compared. For cocaine injection responsive neurons, the initial phase (0-10 minutes) and the decay phase (50-60 minutes) were compared with the pre-injection phase (−10-0 minutes). When analyzing the calcium signal transients of 10-minute windows pre- and post-injection, “actual transient change” was first calculated by subtracting the “pre-injection transients” from the “post-injection transients.” Then transients of the two 10-minute windows, the “pre-injection transients” and “post-injection transients,” were combined to generate “all transients,” and the inter-transient intervals of the “all transients” was determined. The permutation method was used to create 10,000 shuffled distributions of the inter transient intervals, and generate “shuffled pre-transients” and “shuffled post-transients.” Transient number change (“shuffled transient change”) was calculated by subtracting “shuffled pre-transients” from “shuffled post-transients.” The 1^{st} percentile value and 99^{th} percentile value of the “shuffled transient change” was calculated, and neurons with “actual transient change” greater than 99^{th} percentile of the “shuffled transient change” were considered as injection excited neurons, and neurons with “actual transient change” lower than 1^{st} percentile of the “shuffled transient change” were considered as injection inhibited neurons. When analyzing the calcium signal traces of 10-minute windows, the traces (dF over noise) within 10-minute windows were z-scored and divided into ten 1-minute bins, and traces of each bin were averaged. The treatment-responsive neurons were determined by comparing the averaged traces pre- and post-injection using the Wilcoxon signed-rank test. Neurons with significantly higher (p<0.05) values of post-injection traces were classified as injection excited neurons and significantly lower (p<0.05) values of post-injection traces were classified as injection inhibited neurons.

### Calcium Imaging During Cocaine-Conditioned Place Preference

Custom-made two-compartment CPP chambers (50×25×25 cm, L×D×H) were used with visual cues: the floor and the walls of one chamber were decorated with black horizontal stripes and the other chamber was decorated with yellow diagonal stripes. The modified cocaine-CPP consists of three sessions. In the pre-conditioning session, mice were attached with nVoke microscope and allowed to explore the two CPP chambers and to concurrently record calcium activity for 15 minutes. The time spent in different chambers was recorded, the less preferred chamber was assigned as the cocaine chamber and used to condition cocaine injections, while the other chamber was assigned as the saline chamber and used to condition saline injections. In the conditioning session, mice were conditioned for three days to saline injections in the saline chamber in the morning, and cocaine injection (15 mg/kg) in the cocaine chamber in the afternoon (6 hours apart). The mice were confined in the chambers for 30 minutes. Finally, the next day following conditioning, in the post-condition recording session, calcium activity of mice was again recorded while freely exploring the two chambers for 15 minutes. The mouse trajectories were recorded using a webcam (Logitech) with top view, and the time when the mice staying or entering chambers was analyzed using Etho Vision XT 11 (Noldus) and was also manually inspected. During the 15 minute recording windows of the pre-conditioning session and post-conditioning session, mice traveled and stayed between the two chambers, the calcium signal traces and calcium signal transients were aligned with periods that were staying in the two chambers (cocaine chamber and saline chamber, staying time>5 seconds) and entering from one chamber to the other one. A neuron was identified as a cocaine-associated context encoding (CACE) neuron when neuronal activity (calcium signal traces or calcium signal transient frequency) in cocaine chamber-staying periods was significantly different from neuronal activity in saline chamber-staying periods. When analyzing the calcium signal traces, the trace values of each chamber-staying period were first averaged, and then a Mann-Whitney U-test was used to compare the average trace values of multiple saline chamber-staying periods with those in the multiple cocaine chamber-staying periods. When analyzing the calcium signal transients, transient frequency of each chamber-staying period was first calculated by dividing staying time into numbers of transients, and then a Mann-Whitney U-test was used to compare the transient frequencies. Neurons with significantly higher (p<0.05) transient frequency during cocaine chamber staying periods were classified as cocaine chamber excited neurons and significantly lower (p<0.05) transient frequency during cocaine chamber staying periods were classified as cocaine chamber inhibited neurons.

### Behavioral Assays

Mice were habituated in the testing environment for at least 3 days before being subject to behavioral tests to avoid stress. To manipulate the activity of NAc Tac2-expressing neurons by chemogenetics, mice were intraperitoneally (i.p.) injected with clozapine N-oxide (CNO) (Cayman, #16882) at 2 mg/kg (for hM3Dq) or 5 mg/kg (for hM4Di) 20 minutes before behavioral tests. To manipulate the activity of NAc Tac2-expressing neurons by optogenetics, the 473 nm laser power was adjusted to 8˜10 mW at the optic fiber tip, and the laser stimulation protocols were indicated in figure legends. For all experiments, mice with signs of infection, bleeding, or another unhealthy condition after surgery were excluded from behavioral tests, and mice with missed viral injection or implantation, based on brain atlas, were not included in experimental analyses.

### Open Field Test (OFT)

The experiment was conducted in an 8-chamber activity monitor system (Med Associates). Each test chamber (ENV-510 boxes) consists of a 27 cm×27 cm square base and 24-cm walls. For optogenetic experiments, the total duration of the behavioral test was 15 min, which was divided into three 3×5-min epochs (with laser off, on, and off, respectively). The laser pattern is: 473 nm, 20 Hz, 10 ms duration. Activity Monitor software (Med Associates) was used to generate trajectory maps. The distance traveled in the arena, and time spent in the center part of the arena, were recorded and analyzed. After each test session, the chambers were cleaned thoroughly with 20% ethanol.

### Cocaine-Induced Locomotion.

The basal level of locomotion as well as cocaine-induced change of locomotor activity were assessed in the open field arena the same as the OFT. For chemogenetics experiments, 20 minutes after administration of either saline or CNO, mice were individually placed in the center of the test chamber, triggering the infra-red tracking system to record locomotor activity, as measured by the number of beam breaks. For optogenetic experiments, the total duration of the behavioral test was 30 minutes, which was divided into three 5×6-min epochs (with 3-minutes laser on. 3-minutes laser off, respectively). Different groups of mice were balanced across the 8 chambers to avoid potential environmental bias.

### Post-Fasted Food Intake

Mice were firstly fasted overnight (16 hours), and then individually placed in the home cage, regular chow pallets (3 g per pellet) were put in the cage, in the meanwhile, mice received 20 min laser stimulation (4×5 minutes, On-Off-On-Off), and then the remaining food pallets were collected and food intake was measured.

### Elevated Plus Maze (EPM)

EPM was used to measure the anxiety effect. Before the EPM test, mice were brought to the testing room for environmental habituation for at least 30 min. The EPM apparatus is consisted of an elevated platform (80 cm above the floor), with four arms (each arm is 30 cm in length and 5 cm in width), two opposing closed arms with 14 cm walls and two opposing open arms. Mice were attached to the fiber-optic patch cord and were individually placed in the center of the EPM apparatus, towards one of the open arms. For optogenetic experiments, the total duration of the behavioral test was 9 min, which was divided into three 3×3-min epochs (with laser off, on, and off, respectively). The mice trajectories were tracked, and the time spent in the open arms was analyzed using Ethovision XT11 (Noldus).

### Real-Time Place Preference (RTPP)

The mice were tested in a two-chamber cage (60 cm×30 cm×30 cm) without additional contextual cues. Mice were gently put into the middle of the cage at the beginning of the test. Mice would receive laser stimulation when entering one side of the chamber randomly assigned at the beginning of the experiment, the laser pattern was 473 nm, 20 Hz, 8 mW, 10 ms duration. Each mouse was tested for 20 minutes. The laser-coupled side was randomly assigned. Mice tracks were analyzed using Ethovision XT11 (Noldus).

### Cocaine Conditioned Place Preference (Cocaine-CPP)

A two-compartment CPP chamber was used with one compartment with grid rod style floor and the other with mesh style floor (Med Associates). Chamber size is 25×19×17 cm (L×D×H). Mice trajectories were tracked by infra-red photobeam detectors, and the travel distance and the duration the mice spent in the two compartments were recorded. The CPP protocol consists of three sessions that include pre-conditioning, conditioning, and post-conditioning. The baseline preference was measured when mice explore the two chambers for 30 minutes, and mice that showed a strong basis (<25% preference) were excluded from the experiments.

In the conditioning session, for ChR2 optogenetics experiments, the optic fibers were secured to the cannula prior to the experiment. The Tac2-Cre mice were conditioned for two days with no laser/saline injections in one chamber in the morning, and laser/cocaine (15 mg/kg) injection in the other chamber in the afternoon (6 hours apart). The mice were confined in the chamber for 30 minutes, and laser stimulation pattern is: 5×6 minutes (3 minutes On, 3 minutes Off, respectively). For hM3Dq chemogenetics experiments, the Tac2-Cre mice were conditioned for two days to saline/saline injections in one chamber in the morning, and CNO (2 mg/kg)/cocaine (15 mg/kg) injection in the other in the afternoon (30 minutes confinement, 6 hours apart). For hM4Di chemogenetics experiments, a suboptimal cocaine-CPP paradigm was used^{10,46}, where mice received a lower cocaine dose (10 mg/kg), and were conditioned with chambers for 15 minutes. The day after conditioning, mice were tested for place preference during a 30-minutes session where they were allowed to freely explore the two chambers. The time spent in the cocaine-coupled chambers was recorded, and the CPP scores were calculated by subtracting the time spent in the pre-conditioning phase from the time spent in the post-conditioning phase.

### Intravenous (i.v.) Catheterization and Cocaine Self-Administration.

Mice were implanted with indwelling catheters (Instech Laboratories, Inc., PA, USA; Cat #C20PU-MJV1926) into the right jugular vein under a combination of ketamine (100 mg/mg) and xylazine (10 mg/mg i.p.) anesthesia. The catheter was then passed subcutaneously to the back and affixed to a vascular access button (Instech Laboratories, Inc., PA, USA; Cat #VABM1B/25). Mice were treated with analgesic meloxicam (2 mg/kg, s.c.) before and 24 hours after surgery. To avoid clotting and to maintain patency, catheters were flushed daily with heparin (30 IU/ml). Three to four days after surgery, mice were trained to self-administer cocaine intravenously in a modular operant chamber (Med Associate Inc., St. Albans, VT, USA). During the acquisition phase, self-administration was performed under a fixed ratio 1 (FR1) schedule of reinforcement in which an active lever press resulted in a cocaine infusion (1.0 mg/kg/infusion) paired with a tone/cue light conditioned stimulus (CS; 30 seconds). Inactive lever presses had no programmed consequences. Each infusion was followed by a 30 second time-out period during which further active lever presses were recorded but did not result in additional intravenous infusion. All responses were recorded automatically using a computer interface and software from Med Associates (St. Albans, VT, USA). Each session lasted for a maximum of 2 hours or until 20 infusions were taken. The acquisition was defined as intake of at least 6 injections within the session and a 3:1 ratio of active to inactive lever press during two consecutive sessions. Following the acquisition, the dose-response curves were tested in the maintenance phase. Mice were i.p. injected with CNO (2 mg/kg for hM3Dq group and 5 mg/kg for hM4Di group) before behavioral tests, and then were subsequently tested under an FR1 schedule, with different unit doses of cocaine (1.0, 0.33, 0.1 and 0.033 mg/kg/infusion). Dose order was randomly assigned to each animal. Each unit dose was measured for one session, and between the test doses, mice were stabilized with the maintenance dose. Throughout self-administration, the catheter patency was assessed periodically using the intravenous infusion of ketamine. Animals that failed catheter patency test were removed from the study.

### Data Analyses and Statistics

The order of the animals in the behavioral tests was randomly assigned. Investigators were not blinded to experimental conditions. However, all data were collected and analyzed strictly in the same way. No statistical methods were used to predetermine sample sizes, but sample sizes were similar to those reported in previous publications. Individual data points are shown in the bar graphs. Statistical p-values were calculated using Prism 9 (GraphPad) or MATLAB R2019 (Mathworks). Statistical details are indicated in the relevant figure legends. Statistical significance was set as p-value≤0.05.

### REFERENCES

1. J. Cami, M. Farré, Drug addiction. New England Journal of Medicine 349, 975-986 (2003).
2. E. C. O'Connor et al., Accumbal D1R Neurons Projecting to Lateral Hypothalamus Authorize Feeding. Neuron 88, 553-564 (2015).
3. G. Dolen, A. Darvishzadeh, K. W. Huang, R. C. Malenka, Social reward requires coordinated activity of nucleus accumbens oxytocin and serotonin. Nature 501, 179-184 (2013).
4. S. L. Smith-Roe, A. E. Kelley, Coincident activation of NMDA and dopamine D1 receptors within the nucleus accumbens core is required for appetitive instrumental learning. J Neurosci 20, 7737-7742 (2000).
5. A. V. Kravitz, L. D. Tye, A. C. Kreitzer, Distinct roles for direct and indirect pathway striatal neurons in reinforcement. Nat Neurosci 15, 816-818 (2012).
6. K. C. Berridge, M. L. Kringelbach, Pleasure systems in the brain. Neuron 86, 646-664 (2015).
7. S. Ikemoto, J. Panksepp, The role of nucleus accumbens dopamine in motivated behavior: a unifying interpretation with special reference to reward-seeking. Brain Research Reviews 31, 6-41 (1999).
8. V. Pascoli, M. Turiault, C. Luscher, Reversal of cocaine-evoked synaptic potentiation resets drug-induced adaptive behaviour. Nature 481, 71-75 (2012).
9. P. E. Phillips, G. D. Stuber, M. L. Heien, R. M. Wightman, R. M. Carelli, Subsecond dopamine release promotes cocaine seeking. Nature 422, 614-618 (2003).
10. M. K. Lobo et al., Cell type-specific loss of BDNF signaling mimics optogenetic control of cocaine reward. Science 330, 385-390 (2010).
11. P. Robledo, R. MALDONADO-LOPEZ, G. F. Koob, Role of dopamine receptors in the nucleus accumbens in the rewarding properties of cocaine. Annals of the New York Academy of Sciences 654, 509-512 (1992).
12. S. J. Russo et al., The addicted synapse: mechanisms of synaptic and structural plasticity in nucleus accumbens. Trends in neurosciences 33, 267-276 (2010).
13. R. Ito, T. W. Robbins, B. J. Everitt, Differential control over cocaine-seeking behavior by nucleus accumbens core and shell. Nat Neurosci 7, 389-397 (2004).
14. R. N. Cardinal, D. R. Pennicott, C. Lakmali, T. W. Robbins, B. J. Everitt, Impulsive choice induced in rats by lesions of the nucleus accumbens core. Science 292, 2499-2501 (2001).
15. V. Pascoli et al., Contrasting forms of cocaine-evoked plasticity control components of relapse. Nature 509, 459-464 (2014).
16. Y. Zhu, C. F. Wienecke, G. Nachtrab, X. Chen, A thalamic input to the nucleus accumbens mediates opiate dependence. Nature 530, 219-222 (2016).
17. G. D. Gibson et al., Distinct Accumbens Shell Output Pathways Promote versus Prevent Relapse to Alcohol Seeking. Neuron 98, 512-520 e516 (2018).
18. C. Soares-Cunha et al., Nucleus accumbens medium spiny neurons subtypes signal both reward and aversion. Mol Psychiatry 25, 3241-3255 (2019).
19. C. R. Gerfen, D. J. Surmeier, Modulation of striatal projection systems by dopamine. Annu Rev Neurosci 34, 441-466 (2011).
20. A. C. Kreitzer, R. C. Malenka, Striatal plasticity and basal ganglia circuit function. Neuron 60, 543-554 (2008).
21. C. R. Gerfen et al., D1 and D2 dopamine receptor-regulated gene expression of striatonigral and striatopallidal neurons. Science 250, 1429-1432 (1990).
22. S. Yawata, T. Yamaguchi, T. Danjo, T. Hikida, S. Nakanishi, Pathway-specific control of reward learning and its flexibility via selective dopamine receptors in the nucleus accumbens. Proc Natl Acad Sci USA 109, 12764-12769 (2012).
23. Y. M. Kupchik et al., Coding the direct/indirect pathways by D1 and D2 receptors is not valid for accumbens projections. Nat Neurosci 18, 1230-1232 (2015).
24. J. W. Dalley et al., Nucleus accumbens D2/3 receptors predict trait impulsivity and cocaine reinforcement. Science 315, 1267-1270 (2007).
25. S. B. Floresco, The nucleus accumbens: an interface between cognition, emotion, and action. Annu Rev Psychol 66, 25-52 (2015).
26. S. Salgado, M. G. Kaplitt, The Nucleus Accumbens: A Comprehensive Review. Stereotact Funct Neurosurg 93, 75-93 (2015).
27. D. C. Castro, M. R. Bruchas, A Motivational and Neuropeptidergic Hub: Anatomical and Functional Diversity within the Nucleus Accumbens Shell. Neuron 102, 529-552 (2019).
28. H. Yang et al., Nucleus Accumbens Subnuclei Regulate Motivated Behavior via Direct Inhibition and Disinhibition of VTA Dopamine Subpopulations. Neuron 97, 434-449 e434 (2018).
29. K. E. Savell et al., A dopamine-induced gene expression signature regulates neuronal function and cocaine response. Science advances 6, caba4221 (2020).
30. G. Stanley, O. Gokce, R. C. Malenka, T. C. Südhof, S. R. Quake, Continuous and discrete neuron types of the adult murine striatum. Neuron 105, 688-699. e688 (2020).
31. R. Chen et al., Decoding molecular and cellular heterogeneity of mouse nucleus accumbens. Nat Neurosci 24.12, 1757-1771 (2021).
32. L. Yuan, Y. N. Dou, Y. G. Sun, Topography of Reward and Aversion Encoding in the Mesolimbic Dopaminergic System. J Neurosci 39, 6472-6481 (2019).
33. R. Al-Hasani et al., Distinct Subpopulations of Nucleus Accumbens Dynorphin Neurons Drive Aversion and Reward. Neuron 87, 1063-1077 (2015).
34. G. D. Chiara, Nucleus accumbens shell and core dopamine-differential role inbehavior and addiction. Behavioural Brain Research 137 (1-2), 75-114 (2012).
35. C. Soares-Cunha et al., Activation of D2 dopamine receptor-expressing neurons in the nucleus accumbens increases motivation. Nat Commun 7, 11829 (2016).
36. S. Pecina, K. C. Berridge, Hedonic hot spot in nucleus accumbens shell: where do mu-opioids cause increased hedonic impact of sweetness? J Neurosci 25, 11777-11786 (2005).
37. J. W. de Jong et al., A neural circuit mechanism for encoding aversive stimuli in the mesolimbic dopamine system. Neuron 101, 133-151. e137 (2019).
38. X. Xiao et al., A Genetically Defined Compartmentalized Striatal Direct Pathway for Negative Reinforcement. Cell 183, 211-227.e220 (2020).
39. J. He et al., Transcriptional and anatomical diversity of medium spiny neurons in the primate striatum. Curr Biol, 31.24, 5473-5486 (2021).
40. M. D. Scofield et al., The Nucleus Accumbens: Mechanisms of Addiction across Drug Classes Reflect the Importance of Glutamate Homeostasis. Pharmacol Rev 68, 816-871 (2016).
41. L. Mar, F.-C. Yang, Q. Ma, Genetic marking and characterization of Tac2-expressing neurons in the central and peripheral nervous system. Molecular brain 5, 1-11 (2012).
42. M. Benuck, A. Lajtha, M. E. Reith, Pharmacokinetics of systemically administered cocaine and locomotor stimulation in mice. Journal of Pharmacology and Experimental Therapeutics 243, 144-149 (1987).
43. C. Brabant et al., Effects of the H 3 receptor inverse agonist thioperamide on cocaine-induced locomotion in mice: Role of the histaminergic system and potential pharmacokinetic interactions. Psychopharmacology 202, 673-687 (2009).
44. S. Thoeni, M. Loureiro, E. C. O'Connor, C. Lüscher, Depression of Accumbal to Lateral Hypothalamic Synapses Gates Overeating. Neuron 107.1, 158-172 (2020).
45. R. Rodgers, E. M. Nikulina, J. C. Cole, Dopamine D1 and D2 receptor ligands modulate the behaviour of mice in the elevated plus-maze. Pharmacology Biochemistry and Behavior 49, 985-995 (1994).
46. P. C. Keyes et al., Orchestrating Opiate-Associated Memories in Thalamic Circuits. Neuron 107, 1113-1123.e1114 (2020).
47. Zelikowsky, M., et al., The neuropeptide Tac2 controls a distributed brain state induced by chronic social isolation stress. Cell 173.5, 1265-1279 (2018).
48. Andero, R., et al., A role for Tac2, NkB, and Nk3 receptor in normal and dysregulated fear memory consolidation. Neuron 83.2, 444-454 (2014).
49. C. K. Kim et al., A Molecular Calcium Integrator Reveals a Striatal Cell Type Driving Aversion. Cell 183, 2003-2019 e2016 (2020).
50. R. van Zessen, J. Flakowski, C. Lüscher, Dynamic dichotomy of accumbal population activity underlies cocaine sensitization. eLife 10, e66048, (2021).
51. E. S. Calipari et al., In vivo imaging identifies temporal signature of D1 and D2 medium spiny neurons in cocaine reward. Proc Natl Acad Sci USA 113, 2726-2731 (2016).
52. G. Paxinos, K. B. Franklin, Paxinos and Franklin's the mouse brain in stereotaxic coordinates. (Academic press, 2019).
53. P. Zhou et al., Efficient and accurate extraction of in vivo calcium signals from microendoscopic video data. Elife 7, c28728 (2018).
54. J. Friedrich, P. Zhou, L. Paninski, Fast online deconvolution of calcium imaging data. PLOS Computational Biology 13, e1005423 (2017).

### Example 2—Administration of Senktide Attenuates Addiction-Related Behavior

The discovery that activation of the Tac2⁺ neurons in the NAc attenuates drug addiction-related behaviors raised the intriguing possibility that activation of this neuron subtype might be a potential therapeutic strategy for the treatment of drug addicts. To test this possibility, potential strategies were investigated, such as pharmacological and neurosurgical methods, to manipulate Tac2⁺ neurons or Tac2 neuronal pathways in vivo, in order to determine whether the manipulation would lead to addiction behavioral outcomes.

The Tac2 gene encodes the neuropeptide neurokinin B (NKB). NKB has high affinity for neurokinin 3 receptor (NK3R, encoded by the TacR3 gene). The NKB-NK3R (ligand-receptor) signal pathway is functional in both central and peripheral nervous systems and have been implicated in diverse physiological processes, such as reproduction, inflammation, and psychiatric disorders⁵⁵. To evaluate the functional relevance of the NKB-NK3R pathway in addiction, an NK3R agonist, senktide, was systemically administered to mice, and then addiction-related behavioral tests were performed. Injection of senktide reduced cocaine-conditioned place preference (FIG. 12A) and cocaine-induced locomotion (FIG. 12B). These results suggest that pharmacological manipulation of Tac2 neuronal pathways could potentially alleviate drug addiction related behaviors. In addition to pharmacological manipulation, deep brain stimulation (DBS)⁵⁶ could also be used as a possible approach to target Tac2 neuronal population in NAc to achieve desired behavioral outcomes.

### REFERENCES

55. Will Spooren, C. R. a. H. M. (2005). “NK3 receptor antagonists—the next generation of antipsychotics.” Nature Reviews Drug Discovery.
56. Wang, T. R., S. Moosa, R. F. Dallapiazza, W. J. Elias and W. J. Lynch (2018). “Deep brain stimulation for the treatment of drug addiction.” Neurosurgical focus 45 (2): E11.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of treating drug addiction, the method comprising activating neurokinin B (NKB)-expressing neurons in a subject.

2. The method of claim 1, wherein the NKB-expressing neurons are dopamine 1 receptor (D1)-expressing neurons.

3. The method of claim 2, wherein the NKB-expressing neurons are medium spiny neurons.

4. The method of claim 3, wherein the NKB-expressing neurons are located in the nucleus accumbens (NAc) of the subject.

5. The method of any one of claims 1-4, wherein the method comprises administering to the subject an effective amount of an agent for stimulating activity of the NKB-expressing neurons in the subject.

6. The method of claim 5, wherein the agent is a small molecule, a hormone, a protein, a peptide, an aptamer, or a nucleic acid.

7. The method of claim 6, wherein the agent activates a G-protein coupled receptor expressed in NKB-expressing neurons of the subject.

8. The method of claim 7, wherein the agent is an agonist of the G-protein coupled receptor.

9. The method of claim 7 or 8, wherein the G-protein coupled receptor is selected from Neurokinin 3 Receptor (NK3R), Thyrotropin Releasing Hormone Receptor (TRHR), and G-Protein Coupled Receptor 158 (GPR158).

10. The method of claim 9, wherein the G-protein coupled receptor is NK3R and the agent is senktide.

11. The method of claim 6, wherein the agent is a nucleic acid encoding an active form of a G-protein coupled receptor.

12. The method of claim 11, wherein the G-protein coupled receptor is selected from NK3R, TRHR, and GPR158.

13. The method of any one of claims 1-12, wherein the method comprises optogenetically stimulating the activity of the NKB-expressing neurons in the subject.

14. The method of claim 13, wherein the method comprises administering to the subject a vector encoding an optogenetically activated protein and laser-stimulating the activity of the optogenetically activated protein in NKB-expressing neurons of the subject.

15. The method of claim 14, wherein the optogenetically activated protein is light-gated cation channel channelrhodopsin (ChR2) and the laser stimulation is blue light laser stimulation.

16. The method of claim 14 or 15, wherein the vector is administered to the NKB-expressing neurons.

17. The method of any one of claims 14-16, wherein the vector is a viral vector.

18. The method of claim 17, wherein the viral vector is a recombinant adeno-associated virus (rAAV) vector.

19. The method of any one of claims 1-18, wherein the method comprises chemogenetically stimulating the activity of the NKB-expressing neurons in the subject.

20. The method of claim 19, wherein the method comprises administering to the subject a vector encoding a chemogenetically activated protein and an agent sufficient to activate the chemogenetically activated protein in NKB-expressing neurons of the subject.

21. The method of claim 20, wherein the chemogenetically activated protein is a modified human M3 muscarinic receptor (hM3Dq) and the agent is clozapine-N-oxide (CNO).

22. The method of claim 20 or 21, wherein the vector is administered to the NKB-expressing neurons.

23. The method of any one of claims 20-22, wherein the vector is a viral vector.

24. The method of claim 23, wherein the viral vector is a recombinant adeno-associated virus (rAAV) vector.

25. The method of any one of claims 1-24, wherein the method comprises electrically stimulating activity of the NKB-expressing neurons in the subject.

26. The method of claim 25, wherein the method comprises treating NKB-expressing neurons in the subject with deep brain stimulation (DBS).

27. The method of any one of claims 5-24, wherein the administration occurs via injection.

28. The method of claim 27, wherein the administration occurs via intravenous injection, intraperitoneal injection, or intracranial injection.

29. The method of any one of claims 5-28, wherein the method results in increased signaling from NKB-expressing neurons of the NAc to the lateral hypothalamus.

30. The method of any one of claims 5-29, wherein the method results in decreased drug reward behavior in the subject.

31. The method of any one of claims 1-30, wherein the subject is a human subject.

32. The method of any one of claims 1-31, wherein the drug addiction is a drug addiction in which NKB-expressing neuron activity in the subject is reduced.

33. The method of any one of claims 1-32, wherein the drug addiction is selected from nicotine addiction, cocaine addiction, opioid addiction, alcohol addiction, barbiturate addiction, and methamphetamine addiction, or a combination thereof.

34. A composition for use in treating drug addiction in a subject in need thereof, the composition comprising an agent for activating neurokinin B (NKB)-expressing neurons in the subject and a pharmaceutically acceptable excipient.

35. The composition of claim 34, wherein the NKB-expressing neurons are dopamine 1 receptor (D1)-expressing neurons.

36. The composition of claim 35, wherein the NKB-expressing neurons are medium spiny neurons.

37. The composition of claim 36, wherein the NKB-expressing neurons are located in the nucleus accumbens (NAc) of the subject.

38. The composition of any one of claims 34-37, wherein the agent is a small molecule, a hormone, a protein, a peptide, an aptamer, or a nucleic acid.

39. The composition of claim 38, wherein the agent activates a G-protein coupled receptor expressed in NKB-expressing neurons of the subject.

40. The composition of claim 39, wherein the agent is an agonist of the G-protein coupled receptor.

41. The composition of claim 39 or 40, wherein the G-protein coupled receptor is selected from Neurokinin 3 Receptor (NK3R), Thyrotropin Releasing Hormone Receptor (TRHR), and G-Protein Coupled Receptor 158 (GPR158).

42. The composition of claim 41, wherein the G-protein coupled receptor is NK3R and the agent is senktide.

43. The composition of claim 38, wherein the agent is a nucleic acid encoding an active form of a G-protein coupled receptor.

44. The composition of claim 43, wherein the G-protein coupled receptor is selected from NK3R, TRHR, and GPR158.

45. The composition of any one of claims 34-37, wherein the agent comprises a vector encoding an optogenetically activated protein, wherein laser stimulation activates the optogenetically activated protein, thereby activating NKB-expressing neurons of the subject.

46. The composition of claim 45, wherein the optogenetically activated protein is light-gated cation channel channelrhodopsin (ChR2) and the laser stimulation is blue light laser stimulation.

47. The composition of claim 45 or 46, wherein the vector is a viral vector.

48. The composition of claim 47, wherein the viral vector is a recombinant adeno-associated virus (rAAV) vector.

49. The composition of any one of claims 34-37, wherein the agent comprises a vector encoding a chemogenetically activated protein, wherein administration of an agent is sufficient to activate the chemogenetically activated protein, thereby activating NKB-expressing neurons of the subject.

50. The composition of claim 49, wherein the chemogenetically activated protein is a modified human M3 muscarinic receptor (hM3Dq) and the agent is clozapine-N-oxide (CNO).

51. The composition of claim 49 or 50, wherein the vector is a viral vector.

52. The composition of claim 51, wherein the viral vector is a recombinant adeno-associated virus (rAAV) vector.

53. The composition of any one of claims 34-52, wherein the composition is suitable for administration via injection.

54. The composition of claim 53, wherein the injection comprises intravenous injection, intraperitoneal injection, or intracranial injection.

55. The composition of any one of claims 34-54, wherein administration of the composition to the subject results in increased signaling from NKB-expressing neurons of the NAc to the lateral hypothalamus.

56. The composition of any one of claims 34-55, wherein administration of the composition to the subject results in decreased drug reward behavior in the subject.

57. The composition of any one of claims 34-56, wherein the subject is a human subject.

58. The composition of any one of claims 34-57, wherein the drug addiction is a drug addiction in which NKB-expressing neuron activity in the subject is reduced.

59. The composition of any one of claims 34-58, wherein the drug addiction is selected from nicotine addiction, cocaine addiction, opioid addiction, alcohol addiction, barbiturate addiction, and methamphetamine addiction, or a combination thereof.
